(19) United States
(12) Patent Application Publication
(10) Pub. No.: US 2024/0208885 A1
(43) Pub. Date: Jun. 27, 2024

(54) SELECTIVE PHOTOCATALYTIC REDUCTION OF ALKYNES TO ALKENES

(71) Applicant: Northwestern University, Evanston, IL (US)

(72) Inventors: Francesca Arcudi, Evanston, IL (US); Luka Dordevic, Evanston, IL (US); Emily A. Weiss, Evanston, IL (US); Samuel I. Stupp, Evanston, IL (US)

(21) Appl. No.: 18/556,949

(22) PCT Filed: Apr. 28, 2022
(86) PCT No.: PCT/US22/26732

§ 371 Date: Oct. 24, 2023

Related U.S. Application Data
(60) Provisional application No. 63/180,798, filed on Apr. 28, 2021.
(60) Provisional application No. 63/254,395, filed on Oct. 11, 2021.

(51) Int. Cl. C07C 5/09; B01J 31/18; B01J 35/39

(52) U.S. Cl. CPC C07C 5/09; B01J 31/183; B01J 35/39; B01J 2231/645; B01J 2531/845; C07C 2531/22

(57) ABSTRACT

Disclosed herein are compositions and methods for the selective photocatalytic reduction of an alkyne compound to an alkene compound (e.g., acetylene to ethylene).

### CROSS-REFERENCE TO RELATED APPLICATIONS

This application claims priority to and the benefit of U.S. Provisional Patent Application No. 63/180,798, filed on Apr. 28, 2021, and U.S. Provisional Patent Application No. 63/254,395, filed on Oct. 11, 2021, the entire contents of each of which are incorporated herein by reference.

### STATEMENT REGARDING FEDERALLY SPONSORED RESEARCH

This invention was made with government support under DE-SC0000989 awarded by the Department of Energy. The government has certain rights in the invention.

### TECHNICAL FIELD

Disclosed herein are compositions and methods for the selective photocatalytic reduction of alkynes to alkenes, e.g., acetylene to ethylene.

### BACKGROUND

The selective hydrogenation of alkynes to alkenes is an important process in chemical industry, being relevant to the production of commodity chemicals. Ethylene is a versatile chemical intermediate in the petrochemical industry for the preparation of some of the world's most used plastics (polyethylene, polyvinyl chloride, and polystyrene). However, ethylene obtained from steam cracking can contain up to 2% acetylene, which is a poison for the polymerization process, and thus is usually removed. Others have optimized energy-intensive thermochemical routes for the acetylene-to-ethylene conversion, such as the catalytic thermal semi-hydrogenation using H₂ gas feedstock, precious metal catalyst (e.g., palladium), and high temperature (FIG. 1A) (see U.S. Pat. No. 6,054,409, see Borodziński el al. Catal. Rev.-Sci. Eng. 48, 91-144 (2006)).

Over-hydrogenation to ethane is an inherent limitation of precious metal catalysts that achieve high conversion using H₂ as the hydrogen source (Borodziński 2006; Armbrüster et al. Nat. Mater. 11, 690-693 (2012); Wang et al. ACS Catal. 10, 6763-6770 (2020)). Traditional thermal hydrogenation also has disadvantages with respect to safety and sustainability: it is an energy-intensive (high-pressure, high-temperature) thermochemical reaction in an H₂ atmosphere, where the H₂ is usually needed in excess and this excess must be separated to avoid possible thermal run-away processes (Borodziński 2006). Physisorption-based acetylene purification (FIG. 1B) is energy-efficient (Chen. et al. Science 366, 241-246 (2019); Li et al. Science 362, 443-446 (2018)), but wastes the separated acetylene rather than converting it to valuable ethylene.

The mild conditions of electrochemical transformations have been exploited to achieve near 100% conversion of acetylene in the presence of excess of ethylene with 21% energy efficiency, and ethylene selectivity of 90.1% with 0.08% residual hydrogen using 0.5 vol. % C₂H₂, 20 vol. % C₂H₄, Ar balance as gas feed (Shi et al. Nat. Catal. 4, 565-574 (2021)), or ethylene selectivity of 97% with 0.7% residual hydrogen from a C₂H₄ feed with 1.0 vol. % of C₂H₂(Bu et al. Nat. Catal. 4, 557-564 (2021)).

With respect to light-powered hydrogenation, a photothermal system achieved 99% conversion of acetylene in the presence of excess of ethylene and H₂ gas (1 vol. % C₂H₂, 20 vol. % C₂H₄, 20 vol. % H₂, Ar balance) but with a selectivity of only 93.5% (Zhou et al. Adv. Mater. 31, 1-7 (2019)). A photocatalytic system achieved 97% selectivity but with a pure acetylene feed (5 vol. %, He balance) and H₂ gas, and had a lower saturated acetylene hydrogenation yield (˜5%) than the analogous thermal reaction (˜18%) (Swearer et al. Proc. Natd. Acad. Sci. U.S.A 113, 8916-8920 (2016)). However, both of these systems have the disadvantage of requiring the precious metal Pd as the catalyst and an external feed of flammable H₂.

### SUMMARY

In one aspect, disclosed herein is a method of synthesizing an alkene compound from an alkyne compound, comprising: providing a first mixture comprising a cobalt(III) porphyrin compound, a photosensitizer, a reducing agent, and water; adding an alkyne compound to the first mixture to form a reaction mixture; and illuminating the reaction mixture with light.

In some embodiments, the cobalt(III) porphyrin compound is selected from [{meso-tetra(4-sulfonatophenyl)porphyrinato}cobalt(III)] and [{meso-tetra(4-carboxyphenyl)porphyrinato}cobalt(III)]. In some embodiments, the cobalt(III) porphyrin compound is [{meso-tetra(4-sulfonatophenyl)porphyrinato}cobalt(II)]. In some embodiments, the first mixture comprises the cobalt(III) porphyrin compound at a concentration of about 0.10 μM to about 50 μM.

In some embodiments, the photosensitizer is selected from tris(2,2′-bipyridyl)dichlororuthenium(II) and mesoporous graphitic carbon nitride. In some embodiments, the photosensitizer is tris(2,2′-bipyridyl)dichlororuthenium(II). In some embodiments, the first mixture comprises the tris(2,2′-bipyridyl)dichlororuthenium(II) photosensitizer at a concentration of about 50 μM to about 500 μM. In some embodiments, the photosensitizer is mesoporous graphic carbon nitride. In some embodiments, the first mixture comprises about 2.5 mg of the mesoporous graphitic carbon nitride.

In some embodiments, the reducing agent is sodium ascorbate, or a mixture of sodium ascorbate and triethanolamine. In some embodiments, the first mixture comprises the reducing agent at a concentration of about 0.01 M to about 0.50 M.

In some embodiments, the alkyne compound is a C₂-C₄ alkyne and the alkene compound is a C₂-C₄ alkene. In some embodiments, the alkyne compound is acetylene and the alkene compound is ethylene. In some embodiments, the alkyne compound is propyne and the alkene compound is propylene. In some embodiments, the method comprises illuminating the reaction mixture with visible light from a light-emitting diode.

In some embodiments, the first mixture is provided in a reaction vessel, and the step of adding the alkyne compound to the first mixture comprises adding the alkyne compound to the reaction vessel at a pressure of about 1 atm.

In some embodiments, the method is conducted at ambient temperature.

In another aspect, disclosed herein is a composition comprising a cobalt(III) porphyrin compound, a photosensitizer, a reducing agent, and water.

In some embodiments, the cobalt(III) porphyrin compound is selected from [{meso-tetra(4-sulfonatophenyl)porphyrinato}cobalt(III)] and [{meso-tetra(4-carboxyphenyl)porphyrinato}cobalt(III)]. In some embodiments, the cobalt(III) porphyrin compound is [{meso-tetra(4-sulfonatophenyl)porphyrinato}cobalt(III)]. In some embodiments, the composition comprises the cobalt(III) porphyrin compound at a concentration of about 0.10 μM to about 50 μM.

In some embodiments, the photosensitizer is selected from tris(2,2′-bipyridyl)dichlororuthenium(II) and mesoporous graphitic carbon nitride. In some embodiments, the photosensitizer is tris(2,2′-bipyridyl)dichlororuthenium(II). In some embodiments, the composition comprises the tris(2,2′-bipyridyl)dichlororuthenium(II) photosensitizer at a concentration of about 50 μM to about 500 μM. In some embodiments, the photosensitizer is mesoporous graphitic carbon nitride. In some embodiments, the composition comprises about 2.5 mg of the mesoporous graphitic carbon nitride.

In some embodiments, the reducing agent is sodium ascorbate. In some embodiments, the reducing agent comprises a mixture of sodium ascorbate and triethanolamine. In some embodiments, the composition comprises the reducing agent at a concentration of about 0.01 M to about 0.50 M.

In some embodiments, the composition has a pH of about 8.0 to about 11.0.

In some embodiments, the composition further comprises an alkyne compound. In some embodiments, the alkyne compound is a C₂-C₄ alkyne. In some embodiments, the alkyne compound is acetylene or propyne. In some embodiments, the alkyne compound is acetylene.

Other aspects and embodiments of the disclosure will become apparent in light of the following description and drawings.

### BRIEF DESCRIPTION OF THE DRAWINGS

FIGS. 1A-1C show various strategies for producing polymer-grade ethylene. FIG. 1A shows a known process for purifying ethylene from acetylene contaminant: semi-hydrogenation of impurity acetylene using noble-metal catalysts and H₂ at high temperature; FIG. 1B shows a method for capture and separation of acetylene from acetylene/ethylene mixtures using porous materials; and FIG. 1C shows a method according to the present disclosure, with ≥99% selective photoreduction of acetylene to ethylene powered by visible light and water at room temperature

FIGS. 2A-2C show sensitizers and catalysts used for photoreduction of acetylene to ethylene and their performance. FIG. 2A shows [Ru(bpy)₃]²⁺ or mpg-CN photosensitizers and CoTPPS catalyst, with redox potentials shown. FIG. 2B shows TON (C₂H₄) by the [Ru(bpy)₃]²⁺/CoTPPS system containing 1 μM CoTPPS, 50 μM [Ru(bpy)₃]²⁺, 0.1 M NaAsc and 0.1 M NaHCO₃ under C₂H₂ (≥99.5 vol. %) irradiated (450 nm) for 6 h, and with conditions that differ from the optimized conditions, as indicated in the axis labels. The inset shows TON (C₂H₄) and S_{C2H4} as a function of irradiation time by the [Ru(bpy)₃]²⁺/CoTPPS system. FIG. 2C shows TON (C₂H₄) by the mpg-CN/CoTPPS system containing 10 μM CoTPPS, 2.5 mg mpg-CN, 0.05 M NaAsc and 0.2 M TEOA under C₂H₂ (≥99.5 vol. %) irradiated (450 nm) for 12 h, and with conditions that differ from the optimized conditions, as indicated in the axis labels. The inset shows TON (C₂H₄) and S_{C2H4} as a function of irradiation time by the mpg-CN/CoTPPS system. Error bars are calculated from two to three runs; uncertainty is ≤10%.

FIGS. 3A-3E show a mechanistic analysis for the photoreduction of acetylene to ethylene. FIG. 3A shows a proposed mechanism for the photoreduction of acetylene to ethylene by the [Ru(bpy)₃]²⁺/CoTPPS system (Asc is ascorbate, PS is photosensitizer and SD is sacrificial donor). FIG. 3B shows that the Co^{III}-porphyrin is reduced by AscH⁻ to Co^{II}. FIG. 3C shows Stern-Volmer plots for the quenching of the [Ru(bpy)₃]²⁺ emission lifetime by NaAsc or CoTPPS. FIG. 3D shows GC-MS spectra of acetylene (left) feedstock and ethylene (right) product for the irradiated (450 nm) [Ru(bpy)₃]²⁺/CoTPPS system under C₂H₂ (5 vol. %, He balance) in MilliQ H₂O (green) or in D₂O (blue, purple). The shift m/z+2 in H₂O corresponds to incorporation of two hydrogens to produce C₂H₄ when starting with C₂H₂. The shifts m/z+4 in D₂O correspond to incorporation of two deuteriums to produce C₂H₂D₂ when starting with C₂H₂ (blue) or of two deuteriums to produce C₂D₄ when starring with C₂D₂ (purple). FIG. 3E shows experimental gas phase IR spectrum of C₂H₂D₂ product (blue) for the irradiated (450 nm) [Ru(bpy)₃]²⁺/CoTPPS system in D₂O (blue in (d)), compared with the DFT calculated gas-phase spectra (black).

FIG. 4 shows typical GC-MS chromatograms observed before and after photoreduction of the ethylene/acetylene mixture. Gas chromatograms (elution order is C₂H₄. C₂H₆ and C₂H₂) detected with flame ion detection before and after irradiation (450 nm) for 28 h of the [Ru(bpy)₃]²⁺/CoTPPS system containing 25 μM CoTPPS, 500 μM [Ru(bpy)₃]²⁺, 0.1 M NaAsc and 0.1 M NaHCO₃ under C₂H₂/C₂H₄ (1 vol. % C₂H₂, 30 vol. % C₂H₄, He balance) mixture. The inset shows C₂H₂ conversion and S_{C2H4} as a function of irradiation time (h) by the [Ru(bpy)₃]²⁺/CoTPPS system under C₂H₂/C₂H₄ mixture.

FIGS. 5A-D show typical GC-MS chromatograms and mass spectra observed for the photoreduction reaction of acetylene. FIG. 5A: Gas chromatograms (retention times of C₂H₄, C₂H₂ and C₂H₆) with total ion count of C₂H₄ standard (2 vol. %) (green) and C₂H₆ standard (2 vol. %) (green), C₂H₂ standard (5 vol. %) (magenta), and a C₂H₂ (5 vol. %)-purged H₂O solution containing 500 μM [Ru(bpy)₃]²⁺, 10 μM CoTPPS, 0.1 M NaAsc and 0.1 M NaHCO₃ after irradiation at 450 nm (140 mW·cm⁻²) for 18 h (black). The pH was 8.4 before bubbling gas. FIG. 5B: Mass spectrum of C₂H₂. FIG. 5C Mass spectrum of C₂H₄. FIG. 5D: Mass spectrum of C₂H₆.

FIGS. 6A-6B show calibration curves for gas chromatography quantification of products for the photoreduction reaction of acetylene using GC-MS. FIG. 6A: Calibration curve for C₂H₄ with the corresponding coefficient of linear correlation (R²). FIG. 6B: Calibration curve for C₂H₆ with the corresponding coefficient of linear correlation (R²).

FIG. 7 shows photocatalytic performance through variation of pH. TON (C₂H₄) and TON (H₂) by the [Ru(bpy)₃]²⁺/CoTPPS system under C₂H₂ (≥99.5 vol. %) (red and cyan) or under Ar (blue) irradiated at 450 nm (140 mW·cm⁻²) for 6 h containing 50 μM [Ru(bpy)₃]²⁺, 1 μM CoTPPS, 0.1 M NaAsc. The pH measured before bubbling gas is 6.8, 7.4, 8.0 (using 0.1 M Na₂HPO₄/NaH₂PO₄ buffers) or 8.4 (using 0.1 M NaHCO₃). Error bars indicate standard error of the mean, calculated from two to three runs.

FIGS. 8A-8H show typical GC-MS chromatograms and mass spectra of the liquid samples observed for the photoreduction reaction of acetylene. FIG. 8A: GC chromatograms with total ion count of CH₃COOC₂H₅ standard, (CH₃CH₂)₂O standard, HCOOH standard, CH₃CH₂OH standard, CH₃CHO standard, CH₃OH standard, and a C₂H₂ (≥99.5 vol. %)-purged H₂O solution containing 50 μM [Ru(bpy)₃]²⁺, 1 μM CoTPPS, 0.1 M NaAsc and 0.1 M NaHCO₃ after kept in the dark or after irradiation at 450 nm (140 mW·cm⁻²) (black) for 6 h. The pH was 8.4 before bubbling gas. * is N₂ or O₂; ** is CO₂; *** is H₂O; **** is CH₃COCH₃ (contained in C₂H₂≥99.5 vol. %, Airgas). FIGS. 8B-8H show mass spectra of CH₃COOC₂H₅, (CH₃CH₂)₂O. HCOOH, CH₃CH₂OH, CH₃CHO, CH₃OH, and C₂H₂. Note: No H₂O₂ was detected using horse radish peroxidase (HRP) and 3,3′,5,5′-tetramethylbenzidine (TMB) assay.

FIG. 9 shows photocatalytic performance through variation of [[Ru(bpy)₃]²⁺]. Optimizations of TON (C₂H₄) and selectivity for C₂H₄ by the [Ru(bpy)₃]²⁺/CoTPPS system under C₂H₂ (≥99.5 vol. %) irradiated at 450 nm (140 mW·cm⁻²) for 6 h through variation of [[Ru(bpy)₃]²⁺] in the presence of 1 μM CoTPPS, 0.1 M NaAsc and 0.1 M NaHCO₃. The pH measured before bubbling gas is 8.4. Error bars indicate standard error of the mean, calculated from two to three runs.

FIG. 10 shows photocatalytic performance through variation of [CoTPPS]. Optimizations of TON (C₂H₄), amount of C₂H₄ (nmol C₂H₄) produced and selectivity for C₂H₄ by the [Ru(bpy)₃]²⁺/CoTPPS system under C₂H₂ (≥99.5 vol. %) irradiated at 450 nm (140 mW·cm⁻²) for 6 h through variation of [CoTPPS] in the presence of 500 μM [Ru(bpy)₃]²⁺, 0.1 M NaAsc and 0.1 M NaHCO₃. The pH measured before bubbling gas is 8.4. Error bars indicate standard error of the mean, calculated from two to three runs.

FIG. 11 shows spectral features of CoTPPS and [Ru(bpy)₃]²⁺. Normalized absorption spectra of 2.0 mL C₂H₂ (≥99.5 vol. %)-purged H₂O containing CoTPPS (magenta) or [Ru(bpy)₃]²⁺ (blue) in 0.1 M NaAsc and 0.1 M NaHCO₃. Normalized photoluminescence spectrum of 2.0 mL C₂H₂ (≥99.5 vol. %)-purged H₂O containing [Ru(bpy)₃]²⁺ (green) in 0.1 M NaAsc and 0.1 M NaHCO₃. The pH was 8.4 before bubbling gas.

FIGS. 12A-12B show photocatalytic performance through variation of sacrificial donor (SD). FIG. 12A: TON (C₂H₄) by the [Ru(bpy)₃]²⁺/CoTPPS system under C₂H₂ (≥99.5 vol. %) irradiated at 450 nm (140 mW·cm⁻²) for 6 h through variation of 0.1 M SD in the presence of 50 μM [Ru(bpy)₃]²⁺ and 1 μM CoTPPS. Error bars indicate standard error of the mean, calculated from two to three runs (trace C₂H₄ with Na₂SO₃ were detected). FIG. 12B: Absorption spectra of 3.0 mL C₂H₂ (≥99.5 vol. %)-purged H₂O containing 1 μM CoTPPS without (top) and with 1 mM SD and recorded after 3 min stirring. The pH measured before bubbling gas is 10.6 with TEOA, 7.0 with EDTA and 8.4 with the other SDs. The sample with NaAsc contains 0.1 M NaHCO₃. Previous work reported [Co^{III}P(H₂O)(OH)] and [Co^{II}P(OH)] as predominant species at pH=8.4.

FIGS. 13A-13B show photocatalytic performance through variation of TPPS catalyst. FIG. 13A: Structure of the MTPPS (M=H₂, Mn^{III}, Fe^{III}, Co^{III}, Ni^{II}, Cu^{II}, Zn^{II}) catalysts. FIG. 13B: TON (C₂H₄) by the [Ru(bpy)₃]²⁺/MTPPS system under C₂H₂ (≥99.5 vol. %) irradiated at 450 nm (140 mW·cm⁻²) for 6 h through variation of 1 μM MTPPS (M=H₂, Mn^{III}, Fe^{III}, Co^{III}, Ni^{II}, Cu^{II}, Zn^{II}) in the presence of 50 μM [Ru(bpy)₃]²⁺, 0.1 M NaAsc and 0.1 M NaHCO₃. The pH measured before bubbling gas is 8.4. Error bars indicate standard error of the mean, calculated from two to three runs.

FIGS. 14A-14D show evolution of the absorption spectrum of [Ru(bpy)₃]²⁺ and CoTPPS with irradiation time. FIG. 14A: Absorption spectra of 2.0 mL C₂H₂ (≥99.5 vol. %)-purged H₂O containing 10 μM CoTPPS, 0.1 M NaAsc and 0.1 M NaHCO₃ upon irradiation at 450 nm (140 mW·cm⁻²). The pH was 8.4 before bubbling gas. FIG. 14B: Absorption spectra of 2.0 mL C₂H₂ (≥99.5 vol. %)-purged H₂O containing 50 μM [Ru(bpy)₃]²⁺, 0.1 M NaAsc and 0.1 M NaHCO₃ upon irradiation at 450 nm (140 mW·cm⁻²). The pH was 8.4 before bubbling gas. FIG. 14C: Time-dependent absorbance changes at 412 nm for 2.0 mL C₂H₂ (≥99.5 vol. %)-purged H₂O containing 10 μM CoTPPS, 0.1 M NaAsc and 0.1 M NaHCO₃ (red) or at 454 nm for 2.0 mL C₂H₂ (≥99.5 vol. %)-purged H₂O containing 50 μM [Ru(bpy)₃]²⁺, 0.1 M NaAsc and 0.1 M NaHCO₃ (blue) upon irradiation at 450 nm (140 mW·cm⁻²). FIG. 14D: Photographs of the quartz cuvettes with septum cap, used to monitor the absorptions over time, containing either 10 μM CoTPPS, 0.1 M NaAsc and 0.1 M NaHCO₃ (left) or 50 μM [Ru(bpy)₃]²⁺, 0.1 M NaAsc and 0.1 M NaHCO₃ (right).

FIG. 15 shows readdition experiments showing recovery of C₂H₄ production. TON (C₂H₄) by the [Ru(bpy)₃]²⁺/CoTPPS system under C₂H₂ (≥99.5 vol. %) irradiated at 450 nm (140 mW·cm⁻²) as a function of irradiation time containing 50 μM [Ru(bpy)₃]²⁺, 1 μM CoTPPS, 0.1 M NaAsc and 0.1 M NaHCO₃ upon readdition of 50 μM [Ru(bpy)₃]²⁺/1 μM CoTPPS (orange), 50 μM [Ru(bpy)₃]²⁺ (blue), 1 μM CoTPPS (purple), 0.1 M NaAsc (green). The pH measured before bubbling gas is 8.4. Error bars indicate standard error of the mean, calculated from two to three runs.

FIG. 16 shows photocatalytic performance in presence of O₂. TON (C₂H₄) by the [Ru(bpy)₃]²⁺/CoTPPS system under C₂H₂ (≥99.5 vol. %) irradiated at 450 nm (140 mW·cm⁻²) for 6 h containing 50 μM [Ru(bpy)₃]²⁺, 1 μM CoTPPS, 0.1 M NaAsc and 0.1 M NaHCO₃ (red) and with different initial concentration of O₂ added in the headspace (blue) after bubbling gas. The pH measured before bubbling gas is 8.4. Error bars indicate standard error of the mean, calculated from two to three runs.

FIG. 17 shows photocatalytic performance in presence of CO₂. TON (C₂H₄) by the [Ru(bpy)₃]²⁺/CoTPPS system under C₂H₂ (≥99.5 vol. %) irradiated at 450 nm (140 mW·cm⁻²) for 6 h containing 50 μM [Ru(bpy)₃]²⁺, 1 μM CoTPPS, 0.1 M NaAsc and 0.1 M NaHCO₃ (red) and with different initial concentration of CO₂ added in the headspace (blue) after bubbling gas. The pH measured before bubbling gas is 8.4. Error bars indicate standard error of the mean, calculated from two to three runs.

FIG. 18 shows photocatalytic performance in presence of CO. TON (C₂H₄) by the [Ru(bpy)₃]²⁺/CoTPPS system under C₂H₂ (≥99.5 vol. %) irradiated at 450 nm (140 mW·cm⁻²) for 6 h containing 50 μM [Ru(bpy)₃]²⁺, 1 μM CoTPPS, 0.1 M NaAsc and 0.1 M NaHCO₃ (red) and with different initial concentration of CO added in the headspace (blue) after bubbling gas. The pH measured before bubbling gas is 8.4. Error bars indicate standard error of the mean, calculated from two to three runs.

FIG. 19 shows electrochemical characterization of NaAsc. Cyclic voltammogram of 1 mM NaAsc in H₂O containing 0.1 M NaHCO₃ (pH 8.4) supported with 0.1 M KCl and purged with C₂H₂ (≥99.5 vol. %) (red).

FIG. 20 shows electrochemical characterization of [Ru(bpy)₃]²⁺. Square-wave voltammogram of 1 mM [Ru(bpy)₃]²⁺ in H₂O containing 0.1 M NaHCO₃ (pH 8.4) supported with 0.1 M KCl and purged with C₂H₂ (≥99.5 vol. %) (red).

FIG. 21 shows electrochemical characterization of CoTPPS. Cyclic voltammograms of 0.5 mM CoTPPS in H₂O containing 0.1 M NaHCO₃ (pH 8.4) supported with 0.1 M KCl and purged with Ar (black) or C₂H₂ (≥99.5 vol. %) (red). (Inset) Zoomed cyclic voltammograms of 0.5 mM CoTPPS in H₂O containing 0.1 M NaHCO₃ (pH 8.4) supported with 0.1 M KCl and purged with Ar (black) or C₂H₂ (≥99.5 vol. %) (red) and square-wave voltammogram of 0.5 mM CoTPPS in H₂O containing 0.1 M NaHCO₃ (pH 8.4) supported with 0.1 M KCl and purged with C₂H₂ (≥99.5 vol. %) (red dotted line) to better display Co^{II}TPPS/[Co^{I}TPPS]⁻.

FIGS. 22A-C show PL quenching experiments. FIG. 22A: Decay of the PL of [Ru(bpy)₃]²⁺ without (blue) and with added 0.01-0.05 M NaAsc (cyan to green). FIG. 22B: Decay of the PL of [Ru(bpy)₃]²⁺ without (blue) and with added 1-10 μM CoTPPS (purple to pink). FIG. 22C: Modified Stern-Volmer quenching plot of the [Ru(bpy)₃]²⁺ lifetimes as a function of the product of the [Ru(bpy)₃]²⁺ lifetime in absence of quencher (τ₀) with different [CoTPPS] (red) and [NaAsc] (grey) and correspondent bimolecular rate constants and quenching rates.

FIGS. 23A-23B show photoreduction of the ethylene/acetylene mixture. FIG. 23A: C₂H₂ conversion (red), residual C₂H₂ concentration (wine) and selectivity for C₂H₄ (blue) as a function of irradiation time (at 450 nm) by the [Ru(bpy)₃]²⁺/CoTPPS system containing 500 μM [Ru(bpy)₃]²⁺, 1 μM CoTPPS, 0.1 M NaAsc and 0.1 M NaHCO₃ under C₂H₂/C₂H₄ (1 vol. % C₂H₂, 30 vol. % C₂H₄, He balance) mixture using GC-FID. The pH was 8.4 before bubbling with C₂H₂/C₂H₄ mixture. FIG. 23B: Zoomed plot from A to better display the residual C₂H₂ concentration (wine) and the selectivity for C₂H₄ (blue) (108 ppm of C₂H₆ are detected after 28 h of irradiation).

FIG. 24 shows typical GC-FID chromatograms of acetylene observed before and after photoreduction of the ethylene/acetylene mixture. Gas chromatograms (retention time of C₂H₂) with total ion count before (black) and after (red) irradiation (450 nm) for 28 h of the [Ru(bpy)₃]²⁺/CoTPPS system containing 500 μM [Ru(bpy)₃]²⁺, 25 μM CoTPPS, 0.1 M NaAsc and 0.1 M NaHCO₃ under C₂H₂/C₂H₄ (1 vol. % C₂H₂, 30 vol. % C₂H₄, He balance) mixture. The pH was 8.4 before bubbling with C₂H₂/C₂H mixture.

FIGS. 25A-25B show calibration curves for gas chromatography quantification of products for the photoreduction of the ethylene/acetylene mixture using GC-FID. FIG. 25A: Calibration curve for C₂H₂ with the corresponding coefficient of linear correlation (R²). FIG. 25B: Calibration curve for C₂H₆ with the corresponding coefficient of linear correlation (R²).

FIGS. 26A-26D show typical GC-MS chromatograms and mass spectra observed for the photoreduction reaction of propyne. FIG. 26A: Gas chromatograms (retention times of C₃H₆, C₃H₈ and C₃H₄) with total ion count of C₃H₆ standard (2 vol. %) (orange), C₃H₈ standard (2 vol. %) (orange), and a C₃H₄ (5 vol. %) headspace from a sample of H₂O solution containing 50 μM [Ru(bpy)₃]²⁺, 1 μM CoTPPS, 0.1 M NaAsc and 0.1 M NaHCO₃ irradiated at 450 nm (140 mW·cm⁻²) for 18 h (black). The pH was 8.4 before bubbling with He and injecting C₃H₄ (≥99 vol. % Sigma) to reach 5 vol. %. FIGS. 26B-26D: Mass spectra of C₃H₄, C₃H₆, and C₃H₈, respectively.

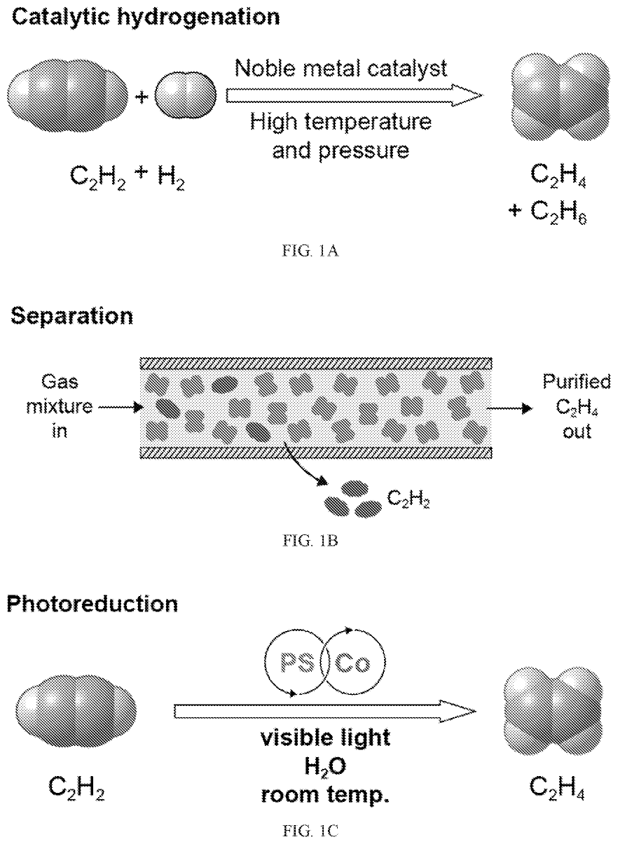

### DETAILED DESCRIPTION

Disclosed herein are compositions and methods for the light-driven photocatalytic reduction of acetylene to ethylene in water and at room temperature, which achieves ≥99% selectivity under both non-competitive (≥99.5 vol. % acetylene; e.g. no ethylene co-feed) and competitive (1 vol. % C₂H₂, 30 vol. % C₂H₄, He balance; e.g. ethylene co-feed) conditions, and near 100% conversion under the latter industrially relevant conditions. This method is based on the use of cobalt salts or complexes as catalyst and a photosensitizer. The method disclosed herein is based on two components: a cobalt porphyrin catalyst and tris(bipyridine)ruthenium or a metal free carbon nitride sensitizer. This light-driven photocatalytic process enables high acetylene-to-ethylene conversion with high selectivity (very low ethane production) using visible light and water rather than heat and hydrogen. Hydrogen gas feedstock, precious metal catalyst, and high temperature are required in benchmark industrial thermal reduction systems. The compositions and methods provide a new, cost-competitive, and sustainable alternative to traditional energy-intensive thermochemical routes.

### Definitions

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

The term “alkane” or “alkane compound,” as used herein, means a straight or branched, saturated hydrocarbon compound. Representative examples of alkane compounds include, but are not limited to, ethane, propane, and butane.

The term “alkene” or “alkene compound,” as used herein, means a straight or branched hydrocarbon compound containing at least one carbon-carbon double bond. The double bond(s) may be located at any positions with the hydrocarbon chain. Representative examples of alkene compounds include, but are not limited to, ethylene, propylene, and butylene (e.g., α-butylene).

The term “alkyne” or “alkyne compound,” as used herein, means a straight or branched hydrocarbon compound containing at least one carbon-carbon triple bond. The triple bond(s) may be located at any positions with the hydrocarbon chain. Representative examples of alkyne compounds include, but are not limited to, acetylene, propyne, and butyne (e.g., 1-butyne).

### Methods

Disclosed herein are methods for synthesizing an alkene compound from an alkyne compound, via a photocatalytic reduction reaction. For example, in some embodiments, acetylene is reduced to ethylene. The energy for the reduction reaction is supplied by light, and no external heating sources, electrical bias, special electrodes, or electrolyte solutions are required. Advantageously, the reduction reaction can be performed at ambient temperature and the hydrogens for the reduction reaction are supplied by water. Experimental results shown in the Examples confirm that the source of ethylene is acetylene, and that protons used for the reduction of acetylene to ethylene are supplied by water molecules.

Accordingly, disclosed herein is a method of synthesizing an alkene compound from an alkyne compound, comprising: providing a first mixture comprising a cobalt(III) porphyrin compound, a photosensitizer, a reducing agent, and water; adding an alkyne compound to the first mixture to form a reaction mixture; and illuminating the reaction mixture with light.

The first mixture includes a cobalt(III) porphyrin compound, such as [({meso-tetra(4-sulfonatophenyl)porphyrinato}cobalt(III)](CoTPPS) or [{meso-tetra(4-carboxyphenyl)porphyrinato}cobalt(III)](CoTCPP). The structures of these compounds are illustrated below. They can be purchased from commercial sources or can be synthesized according to methods known in the art. For example, CoTPPS can be prepared according to: Call et al. ACS Catal. 9, 4867-4874 (2019), which is herein incorporated by reference in its entirety.

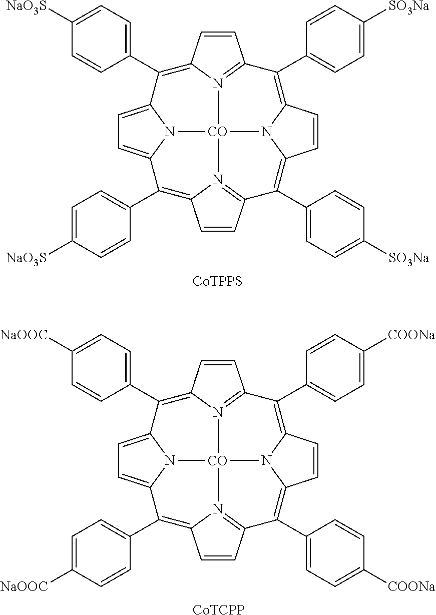

The cobalt(III) porphyrin compound (e.g., CoTPPS or CoTCPP) can be included in the first mixture at a concentration of about 0.10 μM to about 50 μM, or about 1.0 μM to about 25 μM, e.g., about 1.0 μM, about 2.0 μM, about 3.0 μM, about 4.0 μM, about 5.0 μM, about 6.0 μM, about 7.0 μM, about 8.0 μM, about 9.0 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, or about 25 μM.

The first mixture also includes a reducing agent. In some embodiments, the reducing agent is sodium ascorbate. In some embodiments, the reducing agent comprises a mixture of sodium ascorbate and triethanolamine. The reducing agent can be present at a concentration of about 0.01 M to about 0.50 M, or about 0.05 M to about 0.25 M, e.g., about 0.05 M, about 0.06 M, about 0.01 M, about 0.08 M, about 0.09 M, about 0.10 M, about 0.11 M, about 0.12 M, about 0.13 M, about 0.14 M, about 0.15 M, about 0.16 M, about 0.17 M, about 0.18 M, about 0.19 M, about 0.20 M, about 0.21 M, about 0.22 M, about 0.23 M, about 0.24 M, or about 0.25 M. In some embodiments, the reducing agent is present in the first mixture at a concentration of about 0.10 M.

The first mixture also includes a photosensitizer, such as tris(2,2′-bipyridyl)dichlororuthenium(II) or mesoporous graphitic carbon nitride. In some embodiments, the photosensitizer is tris(2,2′-bipyridyl)dichlororuthenium(II). When the photosensitizer is tris(2,2′-bipyridyl)dichlororuthenium(II)), it can be present at a concentration of about 50 μM to about 500 μM, or about 50 μM to about 300 PM, e.g., about 50 μM, about 75 μM, about 100 μM, about 125 μM, about 150 μM, about 175 μM, about 200 μM, about 225 μM, about 250 μM, about 275 μM, about 300 μM, about 350 μM, about 400 μM, about 450 μM, or about 500 μM. In some embodiments, the tris(2,2′-bipyridyl)dichlororuthenium(II) is present at a concentration of about 250 M. In some embodiments, the photosensitizer is mesoporous graphitic carbon nitride. When the photosensitizer is mesoporous graphitic carbon nitride, it can be present in an amount of about 1.0 mg to about 5.0 mg, e.g., about 2.5 mg.

In some embodiments, the first mixture further comprises a buffer, such as a bicarbonate buffer. In some embodiments, the first mixture has a pH of about 8.0 to about 11.0. In some embodiments, the first mixture has a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, or about 11.0. In some embodiments, the first mixture has pH of about 8.4. In some embodiments, the first mixture has pH of about 10.6.

In some embodiments, the alkyne compound used in the methods is a C₂-C₄ alkyne compound, and is reduced to the corresponding C₂-C₄ alkene compound. For example, in some embodiments, the alkyne compound is acetylene and the corresponding alkene compound is ethylene. In other embodiments, the alkyne compound is propyne and the corresponding alkene compound is propylene.

The method includes a step of adding an alkyne compound to the first mixture. In some embodiments, such as when the alkyne compound is in gaseous form, the method can be conducted in a reaction vessel such as a vial, a beaker, a flask, or a chemical reactor (e.g., a research reactor, a commercial reactor, an industrial reactor, or the like), and the alkyne compound can be added to the first mixture by replacing the gas in the headspace above the first mixture with the alkyne compound. In some embodiments, the alkyne compound is added by bubbling it through the first mixture. In some embodiments, the gaseous alkyne compound is added to the reaction mixture at a pressure of about 1 atm.

The methods include a step of illuminating the reaction mixture with visible light. In some embodiments, the method comprises illuminating the mixture with light for about 6 hours to about 96 hours. For example, the mixture may be illuminated for about 6 hours, about 12 hours, 18 hours, about 24 hours, about 30 hours, about 36 hours, about 48 hours, about 60 hours, about 72 hours, or about 96 hours, or any range therebetween. In some embodiments, the light is white light. In some embodiments, the light is blue light (e.g., at about 450 nm). In some embodiments, the method comprises illuminating the reaction mixture with a light-emitting diode (LED), such as a white LED or a blue LED.

In some embodiments, the reaction vessel is of the appropriate scale for the particular application (e.g., <1 L, 1 L, 2 L, 5 L, 10 L, 20 L, 50 L 100 L, 200 L, 500 L, 1000 L, or more, or ranges therebetween). In some embodiments, a chemical reactor is a batch-style reactor, tank reactor, continuous stirred-tank reactor (CSTR), a plug flow reactor, a semi-batch reactor, etc. In some embodiments, a reactor comprises a window or translucent/transparent portion to allow illumination with the appropriate wavelength of light. In some embodiments, a reactor is transparent to the appropriate wavelength of light. In some embodiments, a reactor comprises an internal light source for illumination.

The method is generally conducted at ambient temperature (i.e., room temperature, which is typically about 20° C.) In some embodiments, the method is conducted at a temperature of about 18 to about 24° C.

The methods disclosed herein can produce an alkene compound with high selectivity over the corresponding alkane compound. For example, in some embodiments, the methods produce the alkene compound with greater than about 95% selectivity over the corresponding alkane compound, e.g., greater than about 96%, greater than about 97%, greater than about 98%, or greater than about 99% selectivity. In some embodiments, when the alkyne compound is acetylene, the method produces ethylene with greater than about 95%, greater than about 96%, greater than about 97%, greater than about 98%, or greater than about 99% selectivity over ethane.

In some embodiments, the methods disclosed herein produce an alkene compound from the corresponding alkyne compound with a turnover number (TON) of more than 500, e.g., more than 600, more than 700, more than 800, more than 900, more than 1000, more than 1100, more than 1200, more than 1300, more than 1400, more than 1500, more than 1600, more than 1700, more than 1800, more than 1900, or more than 2000. For example, in some embodiments, the methods disclosed herein produce ethylene from acetylene with a TON of about 1000 to about 2000, or higher.

Alternate methods of carrying out the reaction are also contemplated as part of the present disclosure. For example, in some embodiments, such as those in which [{meso-tetra(4-carboxyphenyl)porphyrinato}cobalt(III)](CoTCPP) is the catalyst, the cobalt(III) porphyrin compound could be anchored to an electrode to exploit the catalytic cycle in a photoelectrochemical cell and the reducing agent may not be required.

### Compositions

Also disclosed herein are compositions comprising a mixture of components that can be used for the photocatalytic reduction of alkynes to alkenes. For example, disclosed herein is a composition comprising a cobalt(III) porphyrin compound, a photosensitizer, a reducing agent, and water.

The composition includes a cobalt(III) porphyrin compound, such as CoTPPS or CoTCPP. In some embodiments, the cobalt (III) porphyrin compound is CoTPPS. The cobalt(III) porphyrin compound can be included in the composition at a concentration of about 0.10 μM to about 50 μM, or about 1.0 μM to about 25 μM, e.g., about 1.0 μM, about 2.0 μM, about 3.0 μM, about 4.0 μM, about 5.0 μM, about 6.0 μM, about 7.0 μM, about 8.0 μM, about 9.0 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, or about 25 μM.

The composition also includes a reducing agent. In some embodiments, the reducing agent is sodium ascorbate. In some embodiments, the reducing agent comprises a mixture of sodium ascorbate and triethanolamine. The reducing agent can be present in the composition at a concentration of about 0.01 M to about 0.50 M, or about 0.05 M to about 0.25 M, e.g., about 0.05 M, about 0.06 M, about 0.01 M, about 0.08 M, about 0.09 M, about 0.10 M, about 0.11 M, about 0.12 M, about 0.13 M, about 0.14 M, about 0.15 M, about 0.16 M, about 0.17 M, about 0.18 M, about 0.19 M, about 0.20 M, about 0.21 M, about 0.22 M, about 0.23 M, about 0.24 M. or about 0.25 M. In some embodiments, the reducing agent is present in the composition at a concentration of about 0.10 M.

The composition also includes a photosensitizer, such as tris(2,2′-bipyridyl)dichlororuthenium(II) or mesoporous graphitic carbon nitride. In some embodiments, the photosensitizer is tris(2,2′-bipyridyl)dichlororuthenium(II). When the photosensitizer is tris(2,2′-bipyridyl)dichlororuthenium(II)), it can be present at a concentration of about 50 μM to about 500 μM, or about 50 μM to about 300 μM, e.g., about 50 μM, about 75 μM, about 100 μM, about 125 μM, about 150 μM, about 175 μM, about 200 μM, about 225 μM, about 250 μM, about 275 μM, about 300 μM, about 350 μM, about 400 μM, about 450 μM, or about 500 μM. In some embodiments, the tris(2,2′-bipyridyl)dichlororuthenium(II) is present at a concentration of about 250 μM. In some embodiments, the photosensitizer is mesoporous graphitic carbon nitride. When the photosensitizer is mesoporous graphitic carbon nitride, it can be present in an amount of about 1.0 mg to about 5.0 mg, e.g., about 2.5 mg. While this amount is used in specific examples disclosed herein, one skilled in the art will appreciate that this amount would be increased as the scale of the reaction increases.

The composition further comprises water. In some embodiments, the composition further comprises a buffer, such as a bicarbonate buffer. In some embodiments, the composition has a pH of about 8.0 to about 11.0. In some embodiments, the composition has a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, or about 11.0. In some embodiments, the composition has pH of about 8.4. In some embodiments, the composition has pH of about 10.6.

In some embodiments, the composition further comprises an alkyne compound. In some embodiments, the alkyne compound is a C₂-C₄ alkyne compound. In some embodiments, the alkyne compound is selected from acetylene and propyne. In some embodiments, the alkyne compound is acetylene.

The following examples further illustrate aspects of the disclosure but, of course, should not be construed as in any way limiting its scope.

### Examples

Safety Warning. It should be noted that acetylene is an extremely flammable gas. Buildup of acetylene vapors can result in fire or explosions if triggered by sparks. Acetylene may displace oxygen and cause rapid suffocation. In a suitable experimental setup, such as the one used for the examples provided herein, the acetylene cylinder was fitted with CGA 510 regulator equipped with flashback arrestor and connected, through stainless steel tubing and flow regulator, to a purging station that was placed inside the fumehood. A soap solution was painted on fittings and joints to inspect for leaks, until no bubble formation was observed. There was no electrical equipment in the fumehood. Additionally, propyne is also a compressed gas that is extremely flammable, may displace oxygen (causing suffocation) and may cause respiratory irritation. In a suitable experimental setup, such as the one used for the examples provided herein, in a fumehood, vials were purged first with helium and then propyne (an aluminum cylinder equipped with stainless steel valve and hose adapter) was injected via gas-tight syringe.

Materials. Sodium ascorbate (NaAsc, Spectrum Chemical, 99%), triethanolamine (TEOA, Sigma-Aldrich, 99.5%), sodium bicarbonate (NaHCO₃, Fisher Chemicals, ACS grade), sodium borohydride (NaBH₄, Sigma-Aldrich, ≥96%), dehydroascorbic acid (DHA, Sigma-Aldrich), 4-hydroxy-TEMPO (Sigma-Aldrich, 97%), tris(2,2′-bipyridyl)dichlororuthenium(II) hexahydrate (Sigma-Aldrich, 99.95%), cobalt nanoparticles (cobalt(II,III) oxide, 99.5%, <50 nm, Sigma-Aldrich), mercury (Hg, Aldrich, ≥99.9%), [{meso-tetra(4-sulfonatophenyl)porphyrinato}copper(II)](Frontier Scientific). [{meso-tetra(4-sulfonatophenyl)porphyrinato}iron(III)] chloride (Frontier Scientific) and [{meso-tetra(4-sulfonatophenyl)porphyrinato}manganese(III)] chloride (Frontier Scientific) were used as received. Ultrapure water (>18.25 MΩcm) was used for the experiments. [{meso-tetra(4-sulfonatophenyl)porphyrinato}cobalt(III)]hydrate (Call, A. et al. Highly Efficient and Selective Photocatalytic CO₂ Reduction to CO in Water by a Cobalt Porphyrin Molecular Catalyst. ACS Catal. 9, 4867-4874 (2019)). [{meso-tetra(4-carboxyphenyl)porphyrinato}cobalt(III)] chloride (Lin et al. Angew. Chemie Int. Ed. 55, 13739-13743 (2016)), [{meso-tetra(4-carboxyphenyl)porphyrinato}nickel(II)](Liu et al. Angew. Chemie Int. Ed. 60, 13463-13469 (2021); Zoltan et al. Spectrochim. Acta Part A Mol. Biomol. Spectrosc. 135, 747-756 (2015)), and mesoporous graphitic carbon nitride (mpg-CN; Ghosh et al. Science 365, 360-366 (2019)) were synthesized according to previously reported procedures.

Photocatalytic Reactions. For photocatalytic experiments, generally, an aqueous solution (2.0 mL) containing CoTPPS catalyst (1-25 μM), sensitizer (50-500 μM [Ru(bpy)₃]²⁺ or 2.5 mg mpg-CN), and sacrificial donor (0.1 M NaAsc or 0.2 M TEOA+0.05 M NaAsc) was purged with acetylene or acetylene/ethylene mixture and illuminated with blue (450 nm) or white LED light (140-200 mW·cm⁻²). Samples were prepared in a 9.0 mL screw cap vial (03-338C, Fisher Scientific) equipped with a micro stir bar (7 mm, Fisher Scientific) and sealed with silicone/PTFE septum (TS-12713. Thermo Scientific) and cap (open top, TS-13216, Thermo Scientific). Vials were then purged, through a flow regulator (at 15 mm), either for (i) 10 minutes with He (or Ar) followed by 5 minutes with C₂H₂ (≥99.5 vol. %. Airgas) or (ii) 15 minutes with He followed by injecting C₃H₄ (350 μL with gas-tight Hamilton syringe of ≥99 vol. % C₃H₄, Sigma-Aldrich) or (iii) 15 minutes with Ar (UHP, Airgas) or (iv) 15 minutes with C₂H₂ (5 vol. %, He balance, Airgas) or (v) 15 minutes with C₂H₄/C₂H₂ mixture (1 vol. % C₂H₂, 30 vol. % C₂H₄, He balance, Airgas) by using steel needles inserted through the septum as inlet (inside the solution) and outlet (to the headspace). After purging for the time specified above, the pressure of the headspace was then equilibrated to 1 atm. The vials were then illuminated using a homebuilt photoreactor made of royal blue (450 nm) LEDs (Cree XLamp XP-E2 Color High Power LED Star, LEDsupply.com) or white light (Cree XLamp XT-E 5000K, LEDsupply.com) with a light intensity of 140 mW·cm⁻² (measured using an Optical Power Meter PM100D with Optical Sensor S120VC from Thorlabs). Each vial was suspended on top of a single LED, equipped with a lens, using a homebuilt sample holder. The vials were continuously stirred at 500 rpm during the irradiation.

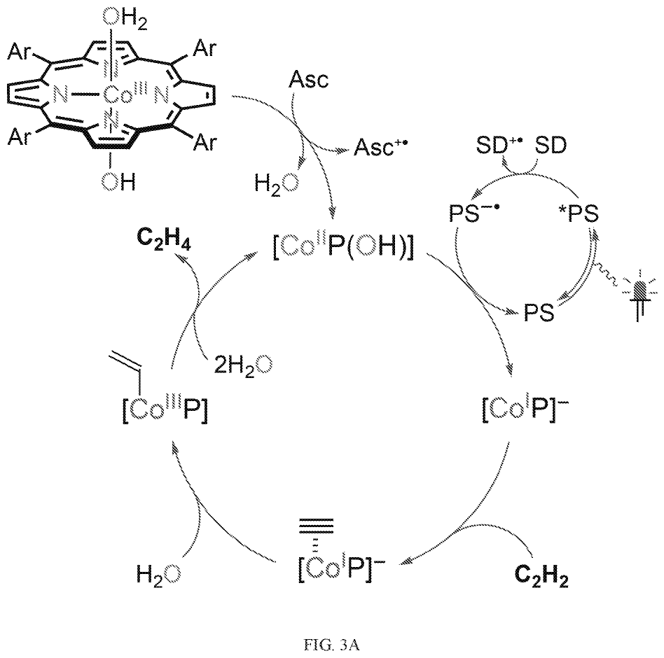

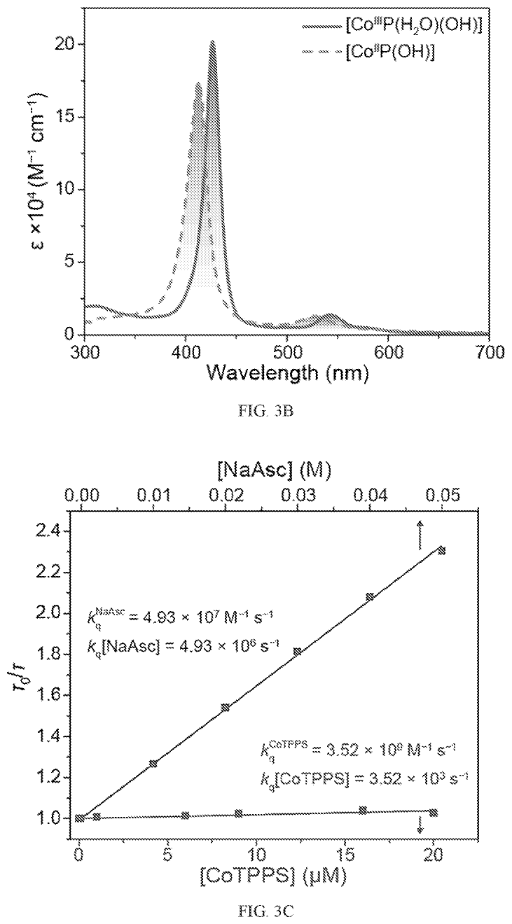

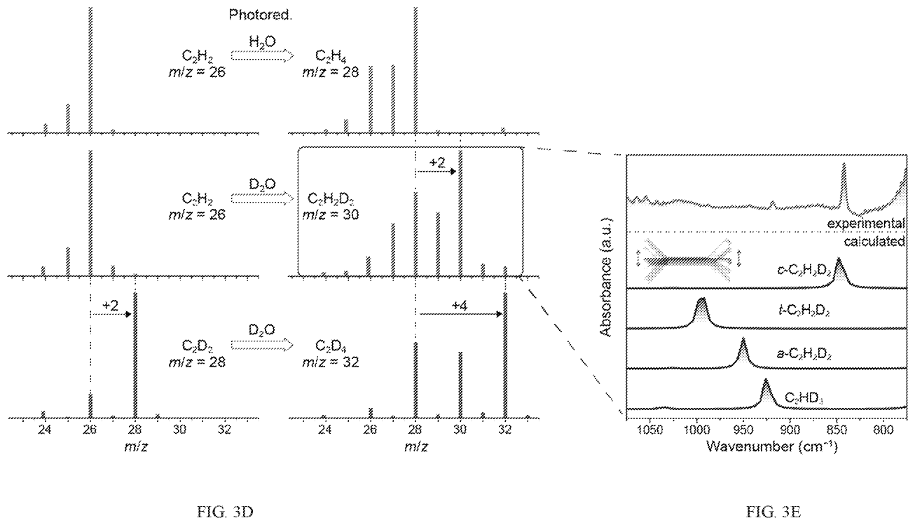

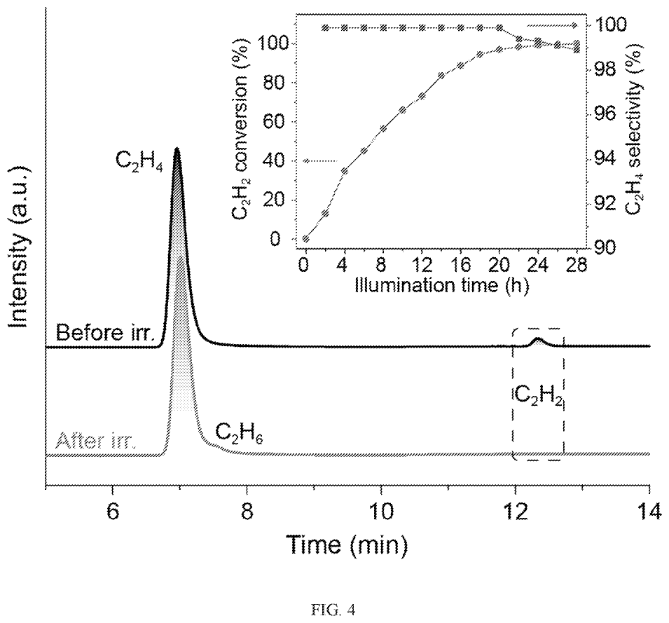

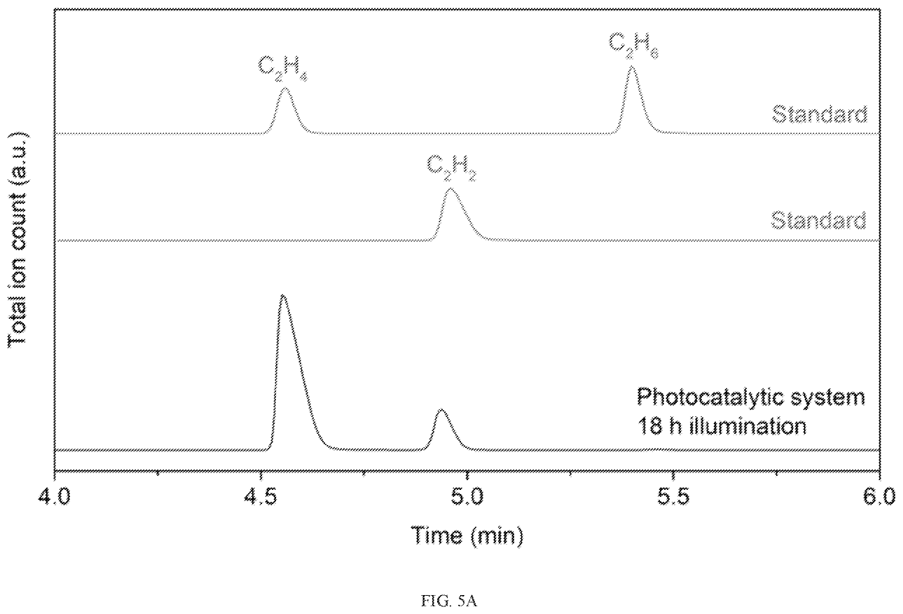

The samples of FIG. 3D were: (i) a 2.0 mL C₂H₂ (5 vol. %, He balance)-purged H₂O solution containing 50 μM [Ru(bpy)₃]²⁺, 1 μM CoTPPS, 0.1 M NaAsc and 0.1 M NaHCO₃ upon irradiation at 450 nm (140 mW·cm⁻²) for 30 minutes. The pH was 6.0 before bubbling with C₂H₂; (ii) a 1.0 mL C₂H₂ (5 vol. %, He balance)-purged D₂O solution containing 100 μM [Ru(bpy)₃]²⁺, 2 μM CoTPPS and 0.2 M NaAsc upon irradiation at 450 nm (140 mW·cm⁻²) for 30 minutes. The pH was 6.0 before bubbling with C₂H₂; (iii) a 2.0 mL C₂H₂ (5 vol. %, He balance)-purged D₂O solution containing 50 μM [Ru(bpy)₃]²⁺, 1 μM CoTPPS, 0.1 M NaAsc and 0.1 M NaHCO₃ that was kept in the dark for 12 hours and then irradiated at 450 nm (140 mW·cm⁻²) for 6 hours. The pH was 8.4 before bubbling with C₂H₂.

The sample of FIG. 4 is a 4.0 mL C₂H₂/C₂H₄ (1 vol. % C₂H₂, 30 vol. % C₂H₄, He balance)-purged H₂O solution containing 500 μM [Ru(bpy)₃]²⁺, 25 M CoTPPS, 0.1 M NaAsc and 0.1 M NaHCO₃ upon irradiation at 450 nm (200 mW·cm⁻²) for 28 h. The pH was 8.4 before bubbling with the C₂H₂/C₂H₄ mixture.

Chromatographic detection of gases. GC-MS experiments were performed on an Agilent Technologies 6850 Network GC system coupled with a 5975C VL MSD with Triple-Axis Detector. The GC was equipped with a HP-PLOT Q column, the inlet temperature was 250° C., the He carrier gas flow was 1.2 mL·min⁻¹ at a pressure of 4.30 psi. For the detection of ethylene, acetylene, and ethane the oven temperature was kept at 45° C. for 4.50 min, and then heated to 200° C. using a 30° C.·min⁻¹ ramp (total run time 9.67 min). For the detection of propylene, propane and propyne, the oven temperature was kept at 45° C. for 4.50 min, and then heated to 220° C. using a 30° C.·min⁻¹ ramp, and kept at 220° C. for 2 min (total run time 12.33 min). Headspace samples were manually injected using gas-tight Hamilton sample-lock syringes (50-250 μL). The calibration curve for C₂H₄ was collected by injecting known quantities of a gas mixture standard containing C₂H₄ (2 vol. % standard, He balance, Airgas). C₂H₄ is reported as “trace” for samples in the photoreduction of C₂H₂ (≥99.5 vol. %, Airgas) that showed ethylene below the intercept of the calibration curve (7 nmol). The calibration curve for C₂H₆ was collected by injecting known quantities of a gas mixture standard containing C₂H₆ (100 and 4200 ppm). The selectivity for C₂H₄ is reported as >99.9% when no quantifiable C₂H₆ and H₂ are detected in the gas chromatograms. TON (C₂H₄) and selectivity for ethylene (S_{C2H4}) for the photoreduction of C₂H₂ (≥99.5 vol. %, Airgas) were calculated as follows:

$${TON}_{{C}_{2} {H}_{4}} = \frac{mol {C}_{2} {H}_{4}}{mol CoTPPS} \\ {S}_{{C}_{2} {H}_{4}} ( \% ) = \frac{mol {C}_{2} {H}_{4}}{mol {C}_{2} {H}_{4} + mol {C}_{2} {H}_{6} + mol {H}_{2}} \times 100$$

A gas mixture standard containing C₃H₆ (2 vol. % standard, He balance, Airgas) and C₃H₈ (2 vol. % standard, He balance, Airgas) was injected as standard for the detection of propylene and propane. Experiments were performed at least in duplicate.

For the detection and quantification of acetylene and ethane in the photoreduction of the C₂H₄/C₂H₂ mixture (1 vol. % C₂H₂, 30 vol. % C₂H₄, He balance, Airgas) a custom-built Shimadzu GC-2014 gas chromatography system equipped with flame ionization detector (FID) was used. The column used was HayeSep T ( 1/16″, 7.5 m), argon carrier gas flowing at 7.5 mL·min⁻¹ min at constant pressure of 2.5-2.7 bars, and the FID detector kept at 250° C. The oven temperature was kept at 35° C. for 9.0 min, and then heated to 85° C. using a 40° C.·min⁻¹ ramp, and kept at 85° C. for 4.0 min (total run time 17.0 min). Calibration curves for C₂H₂ and C₂H₆ were collected by injecting known quantities of a gas mixture standard (TOGAS 4200 ppm standard and 1% C₂H₂ in C₂H₄ and He). Experiments were performed at least in duplicate. The intercept of the calibration curves for C₂H₂ and C₂H₆ crosses zero to guarantee accuracy when the gas concentration is as low as few ppm. Injections were performed via gas-tight Hamilton syringes (10-25 μL). C₂H₂ conversion (C_{C2H2}) and selectivity for ethylene (S_{C2H4}) for the photoreduction of the C₂H₄/C₂H₂ mixture were calculated as follows:

$${C}_{{C}_{2} {H}_{2}} ( \% ) = \frac{{[ {C}_{2} {H}_{2} ]}_{feed} - {[ {C}_{2} {H}_{2} ]}_{x}}{{[ {C}_{2} {H}_{2} ]}_{feed}} \times 100 \\ {S}_{{C}_{2} {H}_{4}} ( \% ) = \frac{{[ {C}_{2} {H}_{2} ]}_{feed} - {[ {C}_{2} {H}_{2} ]}_{x}}{{[ {C}_{2} {H}_{2} ]}_{feed} - {[ {C}_{2} {H}_{2} ]}_{x} + [ {C}_{2} {H}_{6} ]} \times 100$$

where [C₂H₂]_{feed} represents the acetylene concentration in the feed (1×10 ppm) and [C₂H₂]ₓ and [C₂H₆] are the concentrations of acetylene and ethane in the product. The change in ethylene concentration cannot be accurately measured because of the excess of ethylene in the gas feed and therefore is not used for calculating the selectivity.

Analyses of H₂ gas evolved in the headspace during the photocatalysis were performed with a custom-built Shimadzu GC-2014 gas chromatography system equipped with a thermal conductivity (TCD) detector. H₂ production was quantitatively detected using MS-5A ( 1/16″, 2.5 m) and HayeSep T ( 1/16″, 7.5 m) columns. The temperature was held at 100° C. for the TCD detector and at 40° C. for the oven. The carrier gas was argon flowing at 8.5 mL·min⁻¹, at constant pressure of 3.8-4.0 bars. Injections (100 μL) were performed via an autosampler (AOC 6000) equipped with a gas-tight syringe (SGE autosampler syringe). Calibration curve for H₂ was collected by injecting known quantities of H₂ (5 vol. %, standard, Ar balance, Airgas). Experiments were performed at least in duplicate.

Chromatographic detection of liquids. GC-MS experiments were performed on an Agilent Technologies 6850 Network GC system coupled with a 5975C VL MSD with Triple-Axis Detector. The GC was equipped with a HP-PLOT Q column, the inlet temperature was 250° C. the He carrier gas flow was 1.2 mL·min⁻¹ at a pressure of 4.30 psi. For the detection of liquids, the oven temperature was kept at 45° C. for 4.50 min, and then heated to 200° C. using a 30° C.·min⁻¹ ramp, and kept at 200° C. for 7 min (total run time 16.67 min).

H₂O₂ detection. Detection of the hydrogen peroxide (H₂O₂) was performed using horse radish peroxidase (HRP, Sigma-Aldrich) and 3,3′,5,5′-tetramethylbenzidine (TMB) assay in a citrate-acetate buffer as previously reported (Josephy et al. J. Biol. Chem. 257, 3669-3675 (1982). H₂O₂ can be quantified by using extinction coefficient values for the TMB dimer, and cross-checked with a calibration curve made by measuring solutions of known H₂O₂(Sigma-Aldrich) concentration (Jakešová et al. Adv. Funct. Mater. 26, 5248-5254 (2016). Specifically, 20 μL of a TMB DMSO solution (1.0 mg mL⁻¹) was added to 3.0 mL of enzyme buffer solution in a cuvette (one citrate-acetate tablet in 50 mL of mQ H₂O containing 1.70 mg of HRP enzyme), followed by addition of aliquots (50 μL) from the photocatalysis solution (kept under dark or irradiated for 6 h) and the absorbance at 650 nm was monitored by UV-Vis absorbance spectroscopy. No H₂O₂ was detected.

Measurement of quantum yields. The quantum yield of a photochemical process is calculated as the number of defined events occurring per photon absorbed by the system at a specific wavelength. The ΦC₂H₂(%) was therefore calculated according to the following equation:

$${Φ}_{{C}_{2} {H}_{4}} ( \% ) = \frac{number of {C}_{2} {H}_{4} molecules \times 2}{number of photons absorbed} \times 100$$

To calculate the fraction of photons absorbed, the amount of absorbed light was determined at the beginning of the photocatalytic experiments from (at least) three independent readings of the measured power at the top of the reaction vessel (an Optical Power Meter PM100D with Optical Sensor S120VC from Thorlabs was used). The reaction vessel contained a 2.0 mL solution of aq. NaAsc (0.1 M) and aq. NaHCO₃ (0.1 M) to account for the reflection loss at the glass/air interface. The number of photons absorbed was calculated taking the photon wavelength equal to 450 nm, an incident light power of 140 mW·cm⁻² and considering an illuminated area of 1.767 cm². Under these conditions, 2.0 mL of an aqueous solution containing 50 μM [Ru(bpy)₃]²⁺, 1 μM CoTPPS, 0.1 M NaAsc and 0.1 M NaHCO₃ absorbed 63% of incident photons, while 2.0 mL of an aqueous solution containing 500 μM [Ru(bpy)₃]²⁺, 25 μM CoTPPS, 0.1 M NaAsc and 0.1 M NaHCO₃ absorbed 96% of incident photons. The number of molecules of C₂H₄ was determined from the moles of C₂H₄ in the sample headspace (obtained by GC-MS measurements) from three independent experiments (at 6 h of irradiation). The ΦC₂H₄ for the 50 and 500 μM [Ru(bpy)₃]²⁺ samples were calculated to be 0.03% and 0.11%, respectively.

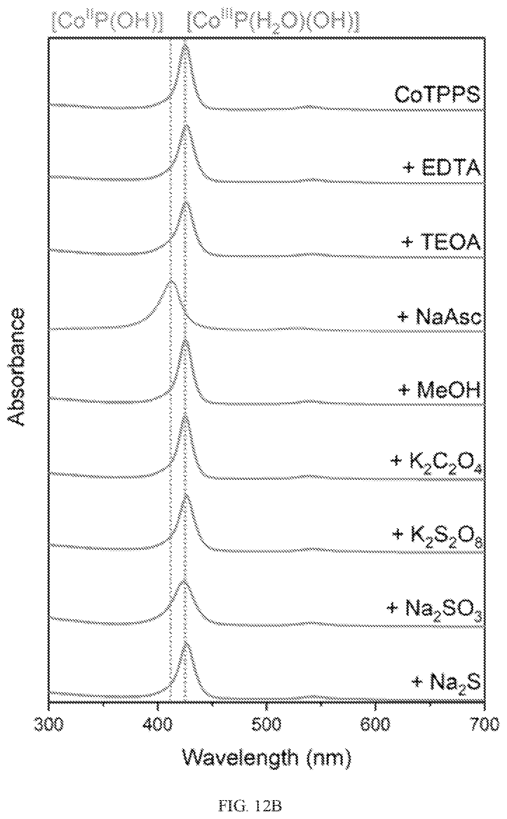

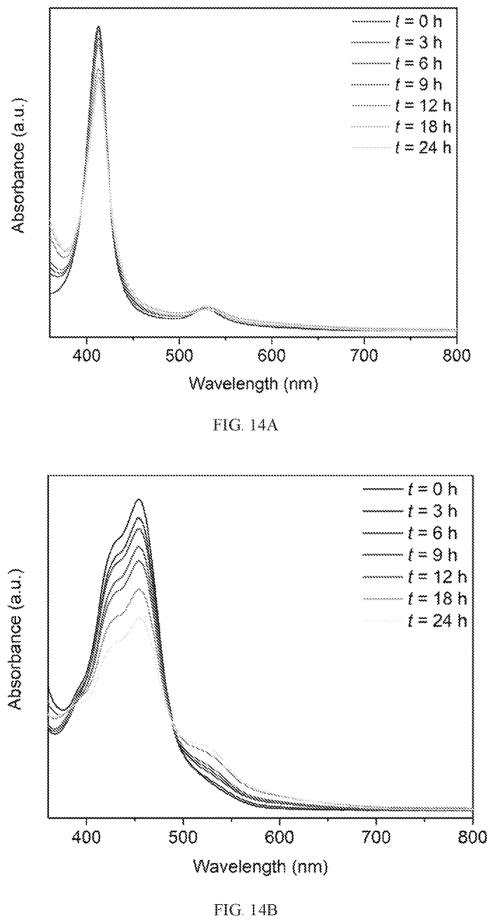

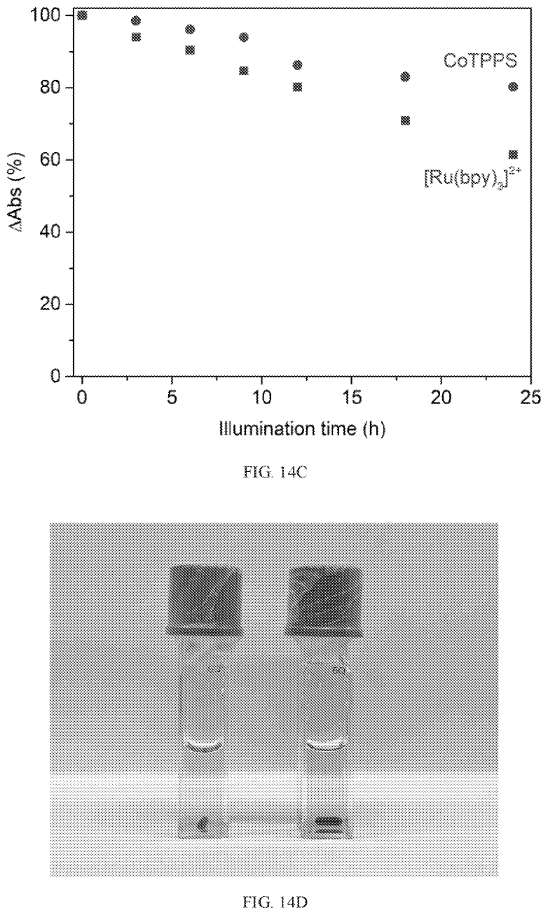

Absorption spectroscopy. UV-Vis absorption spectra were recorded on an Ocean Optics spectrometer (DH-2000-BAL UV-Vis-NIR light source and QE-Pro detector) or a Varian Cary 5000 spectrometer. Sealable quartz cuvettes (Starna Cells with septum cap) equipped with a micro stir bar were used for the reduction of Co(III) porphyrin to Co(II) porphyrin (FIG. 12) and for photostability studies (FIG. 14), and the solutions were prepared and degassed as described for the photocatalytic systems. Irradiation was 450 nm LED (140 mW·cm⁻²). Blank solutions (0.1 M aq. NaHCO₃ and 0.1 M aq. NaAsc) purged with He and/or C₂H₂ were used as baseline.

Fluorescence spectroscopy and fluorescence quenching. Fluorescence spectra were collected on a Fluorolog-3 spectrofluorometer (Horiba Jobin Yvon). For the fluorescence quenching experiments, aqueous solutions of 50 μM [Ru(bpy)₃]²⁺ in 0.1 M NaHCO₃ containing various concentrations of NaAsc or CoTPPS were purged for 10 minutes with He followed by 5 minutes with C₂H₂ (≥99.5 vol. %). The lifetimes were measured in a custom-built microscope equipped with a piezo scanner (NanoPI, Physik Instrumente), an APD detector (MicroPhoton Devices), and a photon counting board (PicoHarp300, PicoQuant) where correlation times between the excitation pulses and detected photons were recorded. The excitation pulses were synchronized from a 450 nm, 70 ps pulsed diode laser at a repetition rate of 500 kHz (Picoquant) and focused with a long working distance objective (0.7 NA, 100×, Mitutoyo), and the detected fluorescence was filtered with a 475 nm long-pass dichroic (Chroma) and a 495 nm long-pass filter (Thorlabs). The quenching rate constant (k_{q}) was calculated according to the Stern-Volmer equation:

$$\frac{{τ}_{0}}{τ} = 1 + {k}_{q} \times {τ}_{0} \times [ Q ]$$

where τ₀ and r are the lifetime of [Ru(bpy)₃]²⁺ in absence (τ0=539 ns) and in presence of quencher (Q) and [Q] is the molar concentration of the quencher ([NaAsc]=0.01-0.05 M. [CoTPPS]=1-20 μM). The measured bimolecular rate constants are k_{q}^{NaAsc}=4.93×10⁷ M⁻¹·s⁻¹ and k_{q}^{CoTPPS}=3.52×10⁹M⁻¹·s⁻¹, which correspond to quenching rate k_{q}[NaAsc]=4.93×10⁶ s⁻¹ and k_{q},[CoTPPS]=3.52×10³s⁻¹ for the concentrations of these species ([NaAsc]=0.1 M, [CoTPPS]=1 μM) used in the catalytic reaction mixtures.

Electrochemical characterization. Cyclic voltammetry (CV) and square-wave voltammetry (SWV) were performed on a CHI660D potentiostat at room temperature, employing a standard three-electrode single-compartment cell: glassy carbon electrode (GCE, CH Instruments, d=3 mm) as working electrode, a Pt wire as counter electrode and Ag/AgCl (3 M aq. KCl) as reference electrode. Working and reference electrodes were polished on a felt pad with 0.3 or 0.05 μm Al₂O₃ suspensions, sonicated in deionized water for about 30 seconds and washed/dried before each experiment; the Pt wire was flame-cleaned. A blank scan was recorded before each sample (scan rate=50 mV·s⁻¹). The aqueous solutions of CoTPPS, NaAsc and [Ru(bpy)₃]²⁺, containing NaHCO₃ (0.1 M) were prepared in mQ H₂O (0.1 M KCl as supporting electrolyte) and degassed with Ar for 15 minutes. After recording the CV (scan rate was 50 mV·s⁻¹) or SWV scans, the same solutions were purged with C₂H₂ (≥99.5 vol. %).

FT-IR spectroscopy. Gas-phase IR measurements were performed on a Nicolet iS50 Fourier Transformed Infrared Spectrometer and a custom-built transmission cell for flowing gases. The gas cell has a gas path of 10 cm, and an internal volume of about 3.5 mL. The cell inlet was modified with an ultra-Torr Swagelok fitting which is capped with a silicone septum. The cell outlet leads to exhaust or a vacuum pump via two on/off valves. The leak rate of the cell, measured in a fully evacuated and closed state, is about 10⁻² torr·sec⁻¹. For the measurements, the cell was first evacuated, and a single beam spectrum was collected. Then, the valve to the pump was closed, and 5.0 mL of sample gas was injected with an air-tight syringe—the sample was a headspace of 30 min photoreduction from a 5.0 mL solution in a 20 mL GC vial containing 100 μM [Ru(bpy)₃]²⁺, 2 μM CoTPPS, 0.2 M NaAsc in D₂O (pH 6.0). The FT-IR spectra were acquired against the reference spectrum obtained in vacuum.

Theoretical calculations. Calculations were carried out with the Gaussian16 package⁹ and all structures were fully optimized without any symmetry constraints at the DFT level. The calculations were performed using the hybrid GGA functional B3LYP, using the 6−31+G(d,p) basis set. Previously, this level of theory has been shown to accurately predict gas-phase IR frequencies (Katari et al. Chem.-A Eur. J. 23, 8414-8423 (2017)). For each stationary point, a vibrational frequency calculation was carried out at the same level of theory to characterize their nature as minima. Lorentzian line shapes, with a full width at half maximum of 8 cm⁻¹, were used to generate the calculated IR spectra. A scaling factor of 0.9759 was used (Katari 2017).

TABLE 1
Photocatalytic and control reactions for C₂H₂ to
C₂H₄ reduction by the [Ru(bpy)₃]²⁺/CoTPPS system

| | | | | TON (selectivity %) | | |
|---|---|---|---|---|---|---|
| Entry | Catalyst | Sensitizer | Reaction conditions | C₂H₂ | C₂H₆ | H₂ |
| 1 | CoTPPS | [Ru(bpy)₃]²⁺ | NaAsc + NaHCO₃ | 915 (99.7) | 3 (0.3) | — |
| 2 | CoTPPS | — | NaAsc + NaHCO₃ | — | — | n.d. |
| 3 | — | [Ru(bpy)₃]²⁺ | NaAsc + NaHCO₃ | — | — | n.d. |
| 4 | CoTPPS | [Ru(bpy)₃]²⁺ | NaHCO₃ | — | — | n.d |
| 5 | CoTPPS | [Ru(bpy)₃]²⁺ | NaAsc + NaHCO₃ | — | — | n.d. |
| 6 | CoTPPS | [Ru(bpy)₃]²⁺ | NaAsc + NaHCO₃ | n.d. | n.d. | 80 |
| 7 | Co NP | [Ru(bpy)₃]²⁺ | NaAsc + NaHCO₃ | 63 (>99) | — | n.d. |
| 8 | CoTPPS | [Ru(bpy)₃]²⁺ | Hg⁰ + NaAsc + NaHCO₃ | 814 (>99) | — | n.d. |
| 9 | CoTPPS | [Ru(bpy)₃]²⁺ | NaAsc + DHA + NaHCO₃ | 228 (>99) | — | n.d. |
| 10 | CoTPPS | — | NaBH₄ + NaHCO; | — | — | n.d. |
| 11 | CoTPPS | — | NaBH₄ + NaHCO₃ | 10 (4.2) | 1 (0.4) | 229 (95.4) |
| 12 | CoTPPS | [Ru(bpy)₃]²⁺ | NaAsc + NaHCO₃ + TEMPO | 930 (>99) | — | n.d. |
| 13 | CoTCPP | [Ru(bpy)₃]²⁺ | NaAsc + NaHCO₃ | 577 (>99) | — | n.d. |

Summary of the reaction conditions used for the photocatalytic and control experiments. In a typical run, 2.0 mL of a C₂H₂ (≥99.5 vol. %)-purged solution (entries 1-5, 7-13) or Ar-purged solution (entry 6) containing catalyst (1 μM CoTPPS (entries 1-2, 4-6, 8-10, 12), 10 μM Co NP < 50 nm (entry 7), 0.5 mM CoTPPS (entry 11), 1 μM CoTCPP (entry 13)), 50 μM [Ru(bpy)₃]²⁺ (entries 1, 3-9, 12-13), Hg⁰ (50,000 eq. vs CoTPPS (entry 8)), NaBH₄ (250 eq. vs CoTPPS (entries 10-11)), 2.5 mM 4-hydroxy-TEMPO (entry 12), NaAsc (0.1M entries 1-3, 5-8, 12-13, 0.09M entry 9), 0.01M DHA (dehydroascorbic acid, entry 9), 0.1M aq. NaHCO₃ (entries 1-13) was irradiated at 450 nm (140 mW · cm⁻²) for 6 h (entries 1-4, 6-9, 12, 13) or was stirred in the dark for 6 h (not irradiated, entries 5, 10, 11).
The pH was measured before bubbling gas as pH = 8.4.
Products were detected via GC-TCD or GC-MS analysis and experiments were performed at least in duplicates (no C₂H₄ in entries 2, 4, 5, 10; trace C₂H₄ in entry 3, no C₂H₆ in entries 2-5, 7-10, 12-13; no H₂ in entry 1). The typical uncertainty for TON values is ≤10%.

TABLE 2
Photocatalytic reaction for C₂H₂ to C₂H₄ reduction through variation
of irradiation time by the [Ru(bpy)₃]²⁺/CoTPPS system

| | |
|---|---|
| Time | TON (selectivity %) |

| Entry | (h) | C₂H₄ | | C₂H₆ |
|---|---|---|---|---|
| 1 | 3 | 347 | (>99.9) | — |
| 2 | 6 | 915 | (99.7) | 3 (0.3) |
| 3 | 9 | 1,241 | (99.8) | 3 (0.2) |
| 4 | 12 | 1,635 | (99.8) | 3 (0.2) |
| 5 | 18 | 1,939 | (99.6) | 8 (0.4) |
| 6 | 24 | 1,967 | (99.4) | 11 (0.6) |

Summary of the reaction conditions used for the photocatalytic experiment.
In a typical run, 2 mL of a C₂H₂ (≥99.5 vol. %)-purged solution containing 1 μM CoTPPS, 50 μM [Ru(bpy)₃]²⁺, 0.1M NaAsc, 0.1M aq. NaHCO₃ was irradiated at 450 nm (140 mW · cm⁻²).
The pH was measured before bubbling gas as pH = 8.4.
Products were detected via GC-MS analysis and experiments were performed at least in duplicates (no C₂H₆ in entry 1).
The typical uncertainty for TON values is ≤10%

TABLE 3
Photocatalytic and control reactions for C₂
H₂ to C₂H₄ reduction by the mpg-CN/CoTPPS system

| | | | | TON (selectivity %) | | |
|---|---|---|---|---|---|---|
| Entry | Catalyst | Sensitizer | Reaction conditions | C₂H₄ | C₂H₆ | H₂ |
| 1 | CoTPPS | mpg-CN | NaAsc + TEOA | 87 (>99.9) | — | — |
| 2 | CoTPPS | mpg-CN | NaAsc + TEOA | 49 (>99) | — | n.d. |
| 3 | CoTPPS | — | NaAsc + TEOA | — | — | n.d. |
| 4 | — | mpg-CN | NaAsc + TEOA | — | — | n.d. |
| 5 | CoTPPS | mpg-CN | — | — | — | n.d. |
| 6 | CoTPPS | mpg-CN | TEOA | 23 (>99) | — | n.d. |
| 7 | CoTPPS | mpg-CN | NaAsc | — | — | n.d. |
| 8 | CoTPPS | mpg-CN | NaAsc + TEOA | — | — | n.d. |
| 9 | CoTPPS | mpg-CN | NaAse + TEOA | n.d. | n.d. | — |

Summary of the reaction conditions used for the photocatalytic and control experiments. In a typical run, 2 mL of a C₂H₂ (≥99.5 vol. %)-purged solution (entries 1-8) or Ar-purged solution (entry 9) containing 10 μM CoTPPS (entries 1-3, 5-9), 2.5 mg mpg-CN (entries 1-2, 4-9), 0.05M NaAsc (entries 1-4, 7-9), 0.2M TEOA (entries 1-4, 6, 8-9), was irradiated at 450 om (140 mW · cm⁻²) for 12 h (entries 1, 3-7, 9) or irradiated with white light (140 mW · cm⁻²) for 12 h (entry 2) or was stirred in the dark for 12 h (not irradiated, entry 8). The pH was measured before bubbling gas as pH = 10.6.
Products were detected via GC-TCD or GC-MS analysis and experiments were performed at least in duplicates (no C₂H₄ in entries 3, 5, 7, 8; trace C₂H₄ in entry 4, no C₂H₆ in entries 1-8; no H₂ in entries 1, 9).
The typical uncertainty for TON values is ≤10%.

TABLE 4
Photocatalytic reaction for C₂H₂ to C₂H₄ reduction through
variation of irradiation time by the mpg-CN/CoTPPS system

| | |
|---|---|
| Time | TON (selectivity %) |

| Entry | (h) | C₂H₄ | C₂H₆ |
|---|---|---|---|
| 1 | 6 | 46 (>99.9) | — |
| 2 | 12 | 87 (>99.9) | — |
| 3 | 24 | 100 (>99.9) | — |
| 4 | 36 | 120 (>99.9) | — |

Summary of the reaction conditions used for the photocatalytic experiment.
In a typical run, 2 mL of a C₂H₂ (≥99.5 vol. %)-purged solution containing 10 μM CoTPPS, 2.5 mg mpg-CN, 0.05M NaAsc, 0.2M TEOA, was irradiated at 450 nm (140 mW · cm⁻²).
The pH was measured before bubbling gas as pH = 10.6.
Products were detected via GC-MS analysis and experiments were performed at least in duplicates (no C₂H₆ in entries 1-4).
The typical uncertainty for TON values is ≤10%.

TABLE 5
Photocatalytic reactions for C₂H₂ to C₂H₄ reduction and H₂ evolution
through variation of pH by the [Ru(bpy)₃]²⁺/CoTPPS system

TON (selectivity %)

| Entry | Gas | pH | C₂H₄ | C₂H₆ | H₂ |
|---|---|---|---|---|---|
| 1 | C₂H₂ | 6.8 | 696 (83.7) | 9 (1.1) | 126 (15.2) |
| 2 | Ar | 6.8 | n.d. | n.d. | 511 |
| 3 | C₂H₂ | 7.4 | 656 (96.2) | 4 (0.6) | 22 (3.3) |
| 4 | Ar | 7.4 | n.d. | n.d. | 102 |
| 5 | C₂H₂ | 8.0 | 669 (99.7) | 2 (0.3) | — |
| 6 | Ar | 8.0 | n.d. | n.d. | 40 |

Summary of the reaction conditions used for the photocatalytic experiments.
In a typical run, 2 mL of a C₂H₂ (≥99.5 vol. %)-purged solution (entries 1, 3, 5) or Ar-purged solution (entries 2, 4, 6) containing 1 μM CoTPPS, 50 μM [Ru(bpy)₃]²⁺, 0.1M NaAsc, 0.1M aq. Na₂HPO₄/NaH₂PO₄¹⁷ was irradiated at 450 nm (140 mW · cm⁻²) for 6 h.
The pH was measured before bubbling gas.
Products were detected via GC-TCD or GC-MS analysis and experiments were performed at least in duplicates (no H₂ in entry 5).
The typical uncertainty for TON values is ≤10%.

TABLE 6
Geometric coordinates for ethylenes reported in FIG. 3E.
α-C₂H₂D₂

| | | | |
|---|---|---|---|
| C | 0.00000000 | −0.66707800 | 0.00000000 |
| H(Iso = 2) | −0.92442900 | −1.23882900 | 0.00000000 |
| C | 0.00000000 | 0.66707800 | 0.00000000 |
| H | 0.92442900 | 1.23883100 | 0.00000000 |
| H(Iso = 2) | 0.92442000 | −1.23884300 | 0.00000000 |
| H | −0.92442200 | 1.23883800 | 0.00000000 |

C₂HD₃

| | | | |
|---|---|---|---|
| C | 0.00000000 | −0.66707800 | 0.00000000 |
| H | −0.92442900 | −1.23882900 | 0.00000000 |
| C | 0.00000000 | 0.66707800 | 0.00000000 |
| H(Iso = 2) | 0.92442900 | 1.23883100 | 0.00000000 |
| H(Iso = 2) | 0.92442000 | −1.23884300 | 0.00000000 |
| H(Iso = 2) | −0.92442200 | 1.23883800 | 0.00000000 |

cis-C₂H₂D₂

| | | | |
|---|---|---|---|
| C | 0.00000000 | −0.66707800 | 0.00000000 |
| H(Iso = 2) | −0.92442900 | −1.23882900 | 0.00000000 |
| C | 0.00000000 | 0.66707800 | 0.00000000 |
| H | 0.92442900 | 1.23883100 | 0.00000000 |
| H | 0.92442000 | −1.23884300 | 0.00000000 |
| H(Iso = 2) | −0.92442200 | 1.23883800 | 0.00000000 |

trans-C₂H₂D₂

| | | | |
|---|---|---|---|
| C | 0.00000000 | −0.66707800 | 0.00000000 |
| H(Iso = 2) | −0.92442900 | −1.23882900 | 0.00000000 |
| C | 0.00000000 | 0.66707800 | 0.00000000 |
| H(Iso = 2) | 0.92442900 | 1.23883100 | 0.00000000 |
| H | 0.92442000 | −1.23884300 | 0.00000000 |
| H | −0.92442200 | 1.23883800 | 0.00000000 |

### Results and Discussion

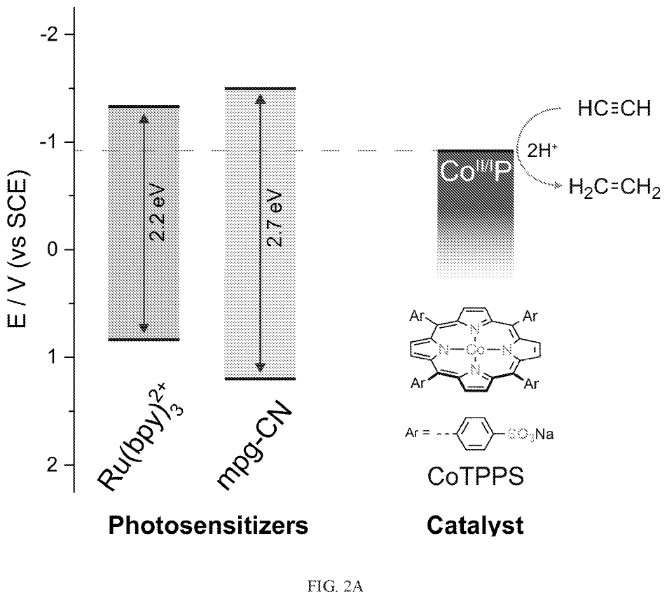

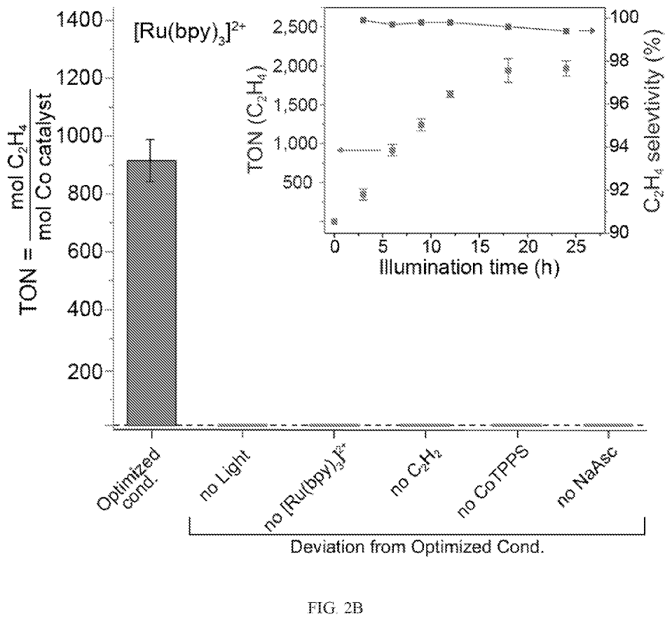

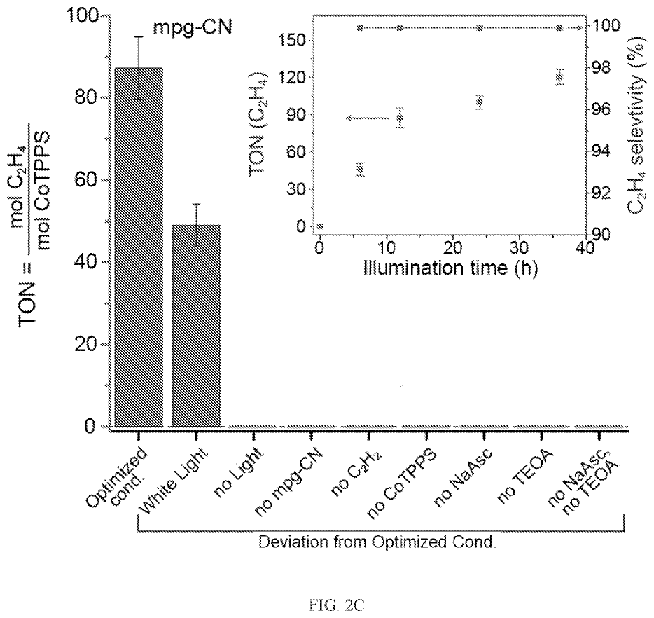

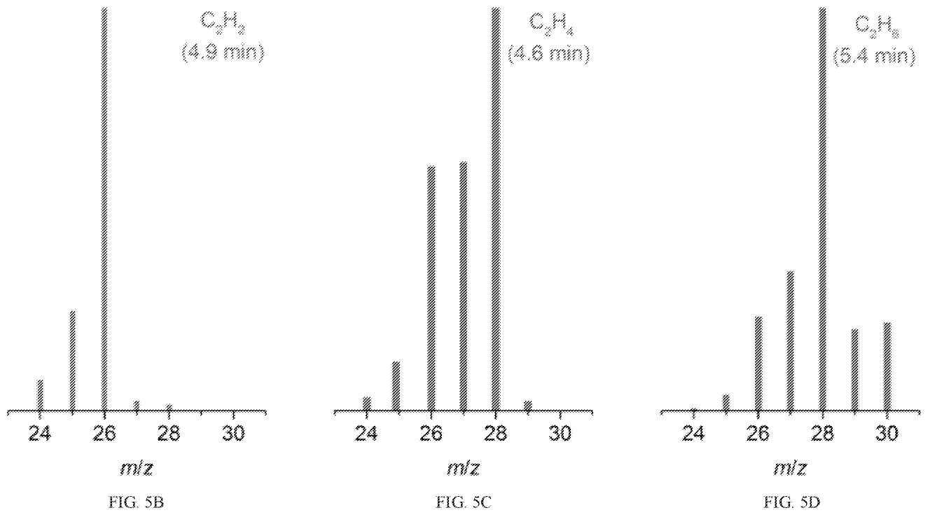

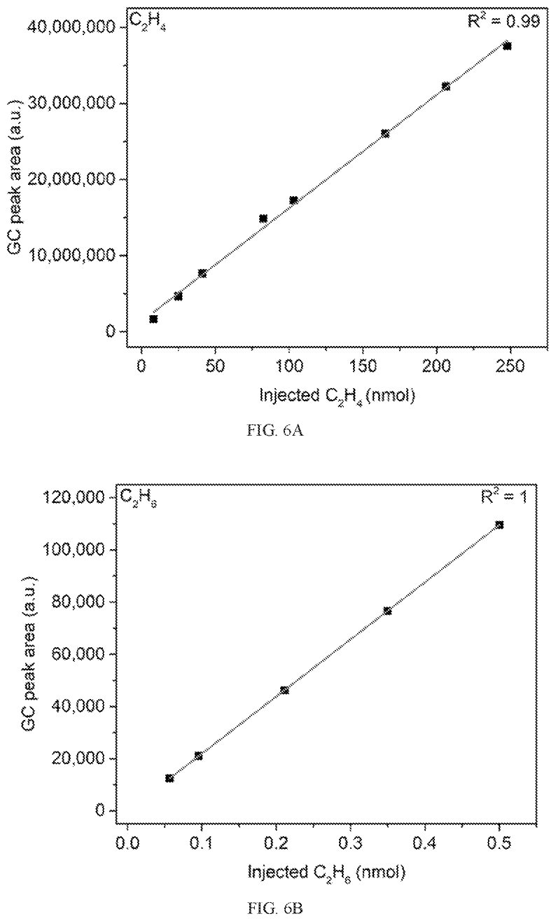

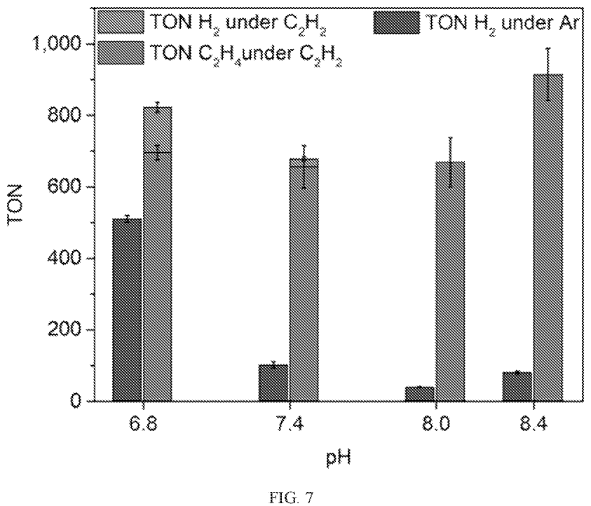

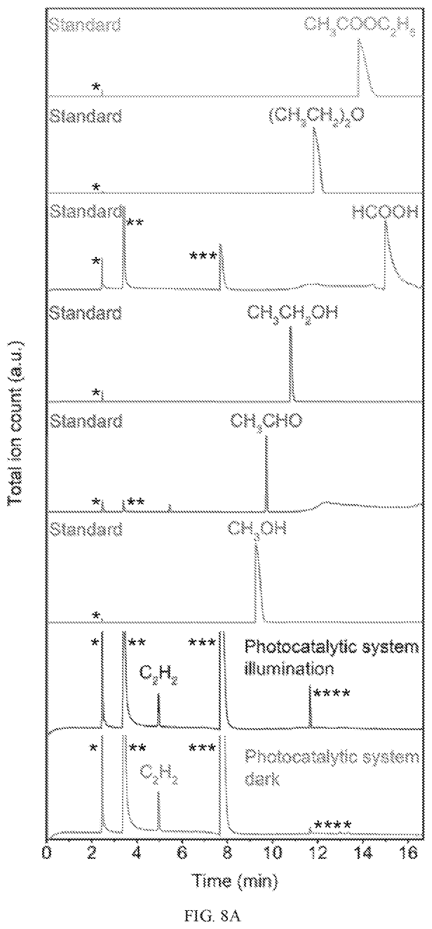

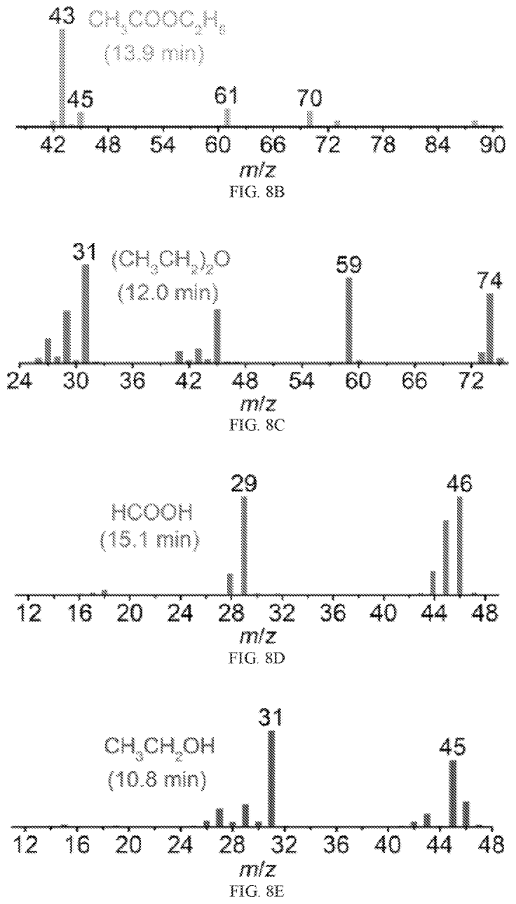

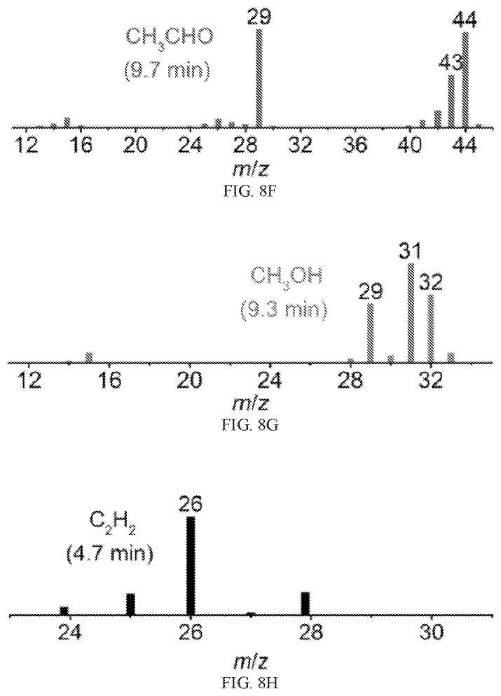

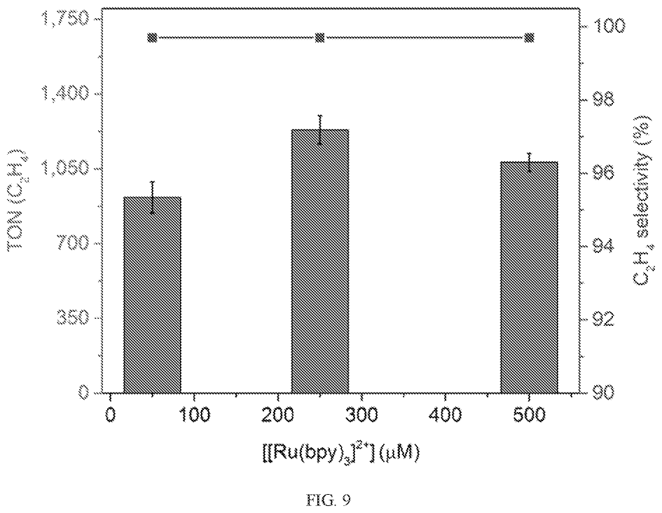

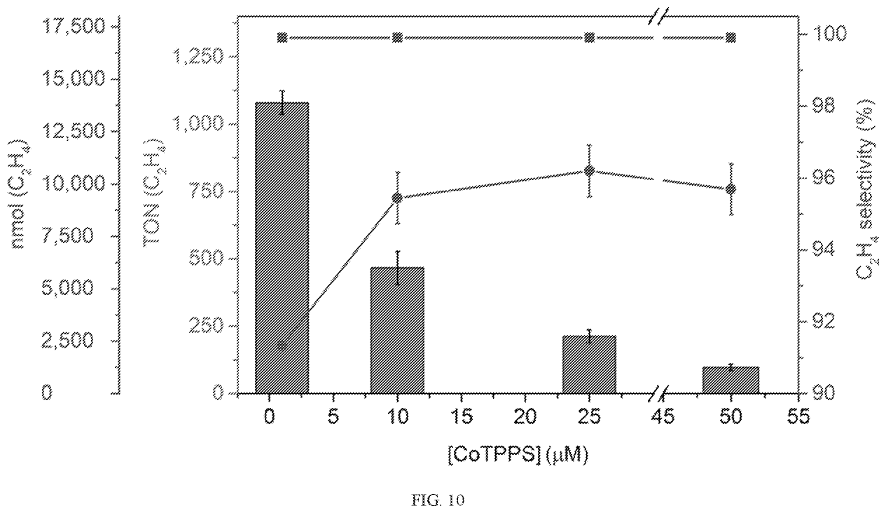

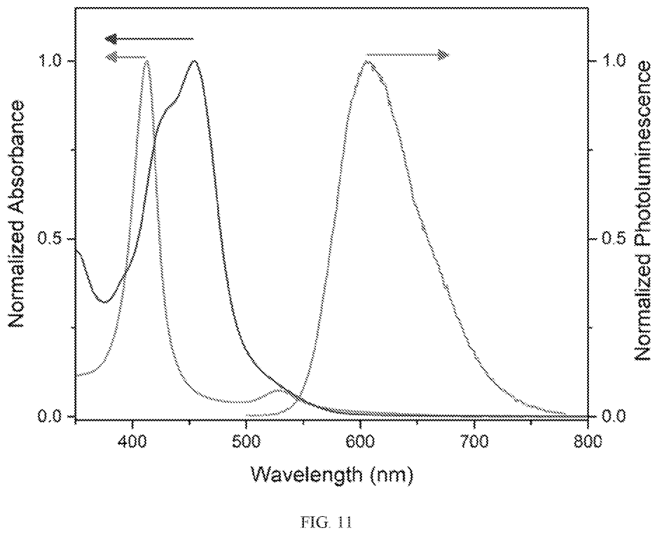

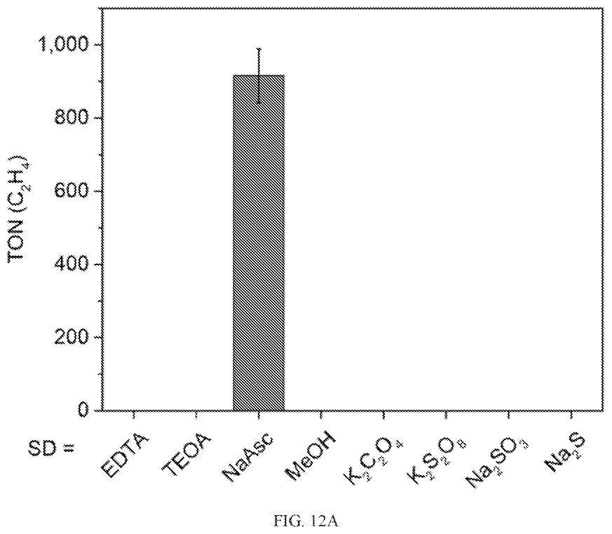

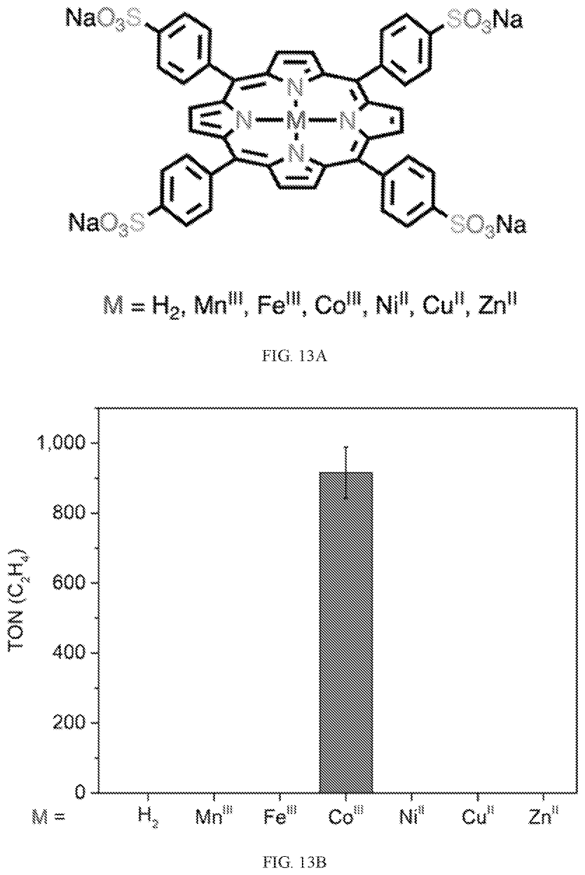

The three-component catalytic reaction mixture used herein contains 1 μM CoTPPS catalyst, 50 μM [Ru(bpy)₃]²⁺ sensitizer, and 0.1 M sodium ascorbate (NaAsc) sacrificial reductant in an aqueous bicarbonate buffer (pH 8.4, in order to disfavor competitive proton reduction). In a typical run, 2.0 mL of this mixture (aq) was illuminated under 1 atm C₂H₂ (≥99.5 vol. %) using a 450 nm light-emitting diode (LED, 140 mW·cm⁻²). Illumination for 24 h produced C₂H₄ with selectivity for ethylene over ethane (S_{C2H4}) of 99.4% and TON=1,967, which confirms the catalytic nature of the reaction (inset in FIG. 2B, FIGS. 5-6, Table 1, entry 1, and Table 2). No other gases (e.g., H₂) or liquid products were detected (FIG. 7, FIG. 8, and Table 1, entry 1). Upon increasing the concentration of [Ru(bpy)₃]²⁺ by a factor of five to 250 μM, the TON (C₂H₄) increased by ˜35% with S_{C2H4}=99.7% after 6 h of illumination; further increase of [[Ru(bpy)₃]²⁺] did not increase the amount of produced C₂H₄ (FIG. 9). The amount of C₂H₄ produced increased with the concentration of catalyst and stopped increasing above [CoTPPS]˜25 μM (FIG. 10 and FIG. 11). No C₂H₄ is produced in the absence of sensitizer, catalyst, sacrificial donor, light or C₂H₂ feedstock (FIG. 2b and Table 1, entries 2-6). Additionally, no C₂H₄ is produced in the presence of other commonly employed sacrificial donors (FIG. 12) or of metal-free or other metals porphyrins (FIG. 13). When photoreduction reaction was performed with cobalt nanoparticles (as opposed to CoTPPS), the TON decreased dramatically, and when mercury was added to the CoTPPS system, the TON was not meaningfully affected (Table 1, entries 7 and 8). These control experiments unequivocally prove that this reaction is primarily homogeneous and photocatalytic, powered by [Ru(bpy)₃]²⁺-sensitized CoTPPS.

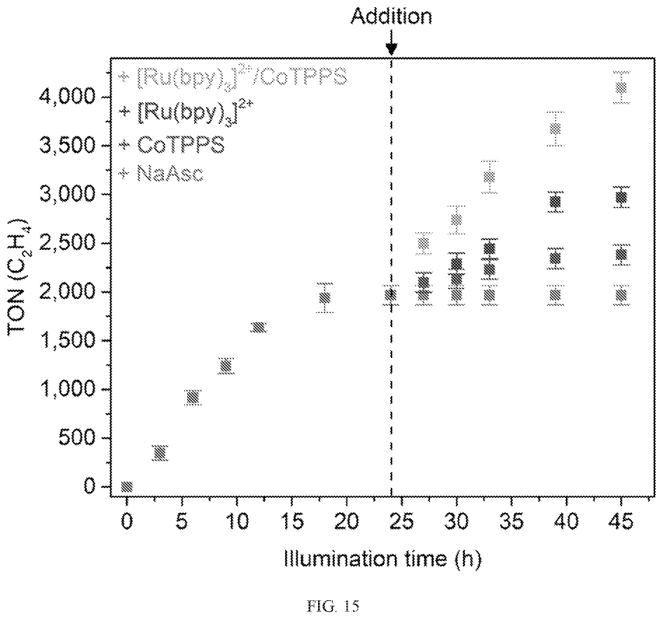

Ethylene production saturated after 24 h (inset in FIG. 2B) due to degradation of both [Ru(bpy)₃]²⁺ and CoTPPS (FIG. 14), probably by the oxidized sacrificial reductant, dehydroascorbic acid, which also inhibits catalysis when added directly to the reaction mixture (Table 1, entry 9). Accordingly, experiments show recovery of the initial activity by re-addition of the initial amount of [Ru(bpy)₃]²⁺ and CoTPPS, partial recovery of the initial activity by re-addition of the initial amount of either [Ru(bpy)₃]²⁺ or CoTPPS, and no recovery of the initial activity by re-addition of the initial amount of NaAsc only (FIG. 15).

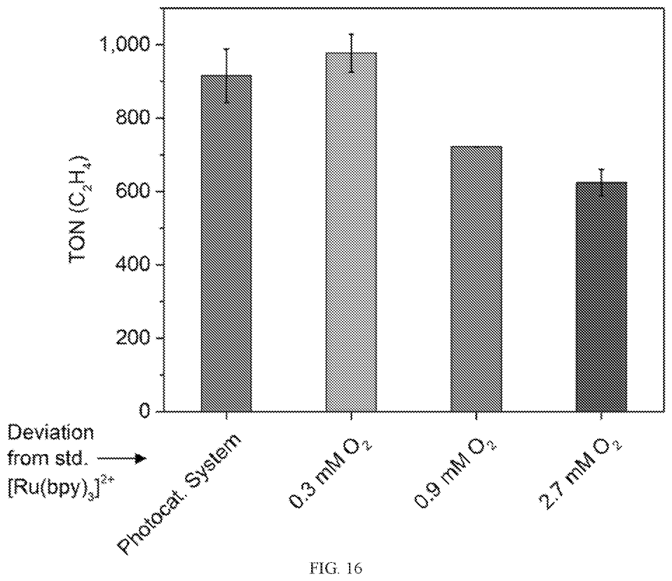

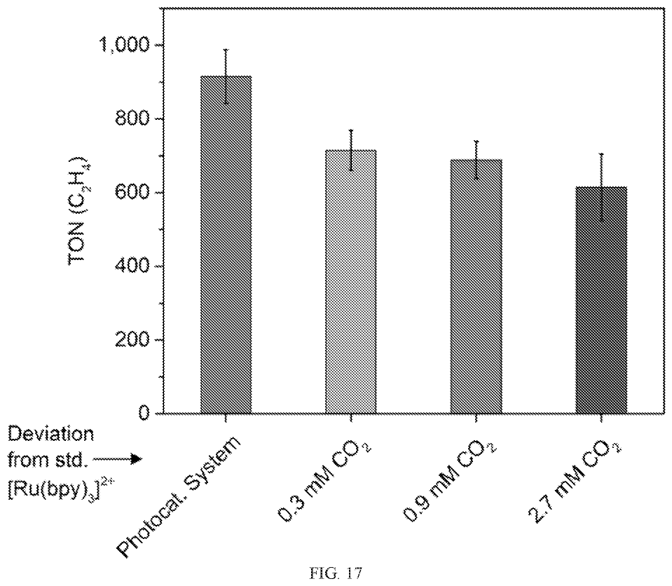

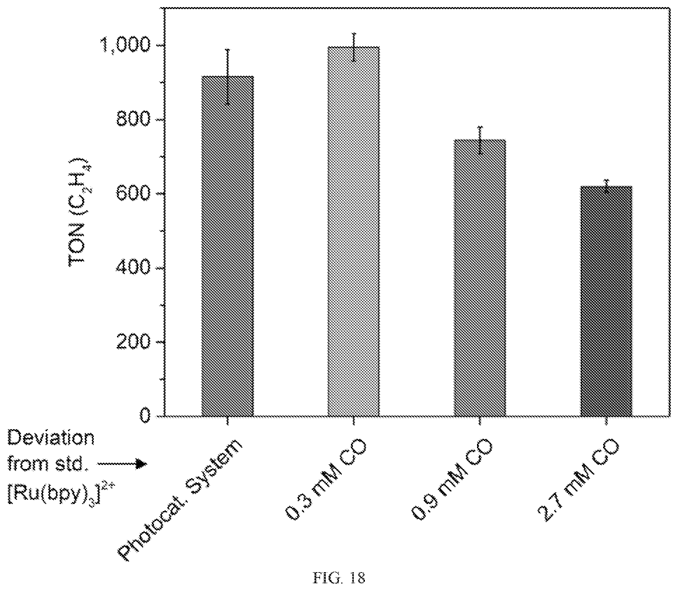

The photocatalytic activity of this acetylene reduction system was tolerant to presence of various gases, including O₂. CO₂, and CO (FIGS. 16-18), the latter two of which are typically present as impurities. CO absorbs to the active sites of conventional hydrogenation catalysts and acts as an inhibitor; tolerance to these adventitious adsorbates highlights this system's potential advantages over traditional catalysts in an industrial setting.

The [Ru(bpy)₃]²⁺ photosensitizer can be replaced with the organic semiconductor mpg·CN, which is a broadband absorber and can be prepared at only a few dollars per kg from readily available starting material. A system containing 10 μM CoTPPS, 2.5 mg mpg-CN and 0.05 M NaAsc+0.2 M TEOA under C₂H₂ (≥99.5 vol. %) in water (pH 10.6) produced C₂H₄ with TON=87, S_{C2H4}>99.9% after 12 h of illumination with a 450 nm LED, and TON=49, S_{C2H4}>99% after 12 h of illumination with white light irradiation (140 mW·cm⁻²) (FIG. 2C, Tables 3 and 4).

A major advantage of the CoTPPS system with respect to sustainability is that it does not require an external feed of H₂, the source of protons/hydrogens was further investigated. The three-component system did not evolve any detectable H₂ (FIG. 7 and Table 1, entry 1), so it is extremely unlikely that in situ production of H₂ is a source of hydrogen here. When experiments were conducted in H₂O and using C₂H₂ (5 vol. %, He balance) as feedstock, gas chromatography/mass spectrometry (GC-MS) analysis identified C₂H₄ (m/z=28) as the reaction product (FIG. 3D). When the photoreduction was instead performed in D₂O solvent, the formation of C₂D₄ (m/z=32) from C₂D₂ (m/z=28) was observed (FIG. 3D), produced by exchange between the feedstock C₂H₂ and D₂O, which was pre-equilibrated before illumination. These two experiments prove unambiguously that not only is acetylene the precursor for the observed C₂H₄, but also the protons added to make the C₂H₄ reduction product originate from the water solvent.

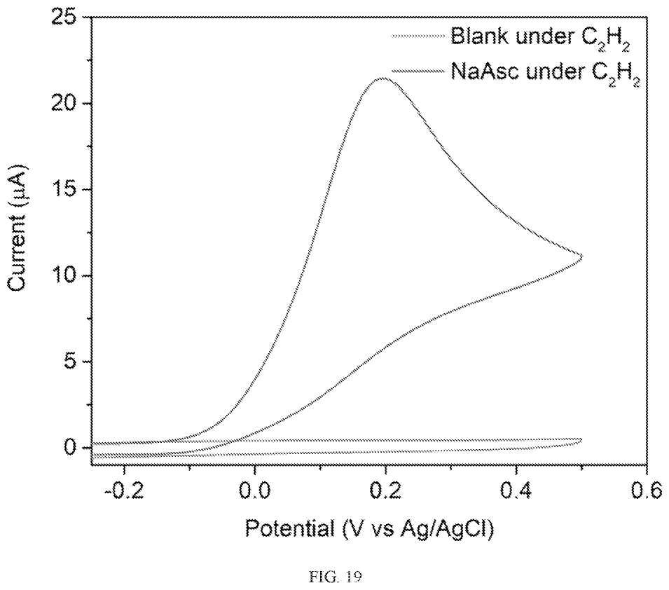

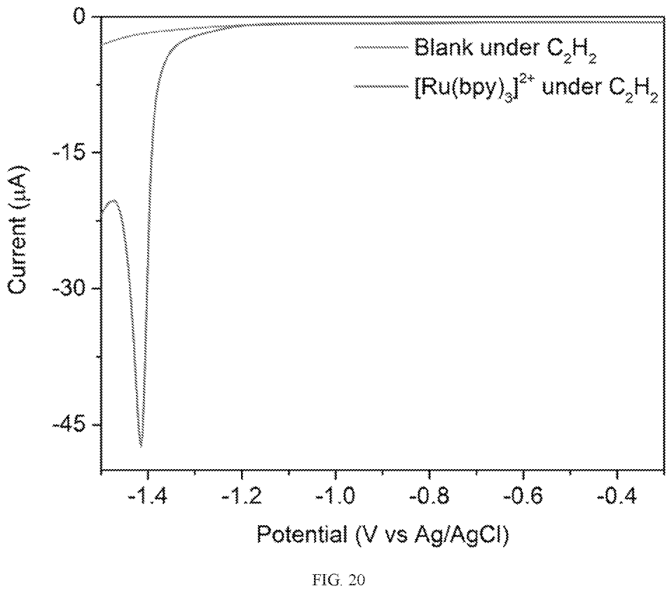

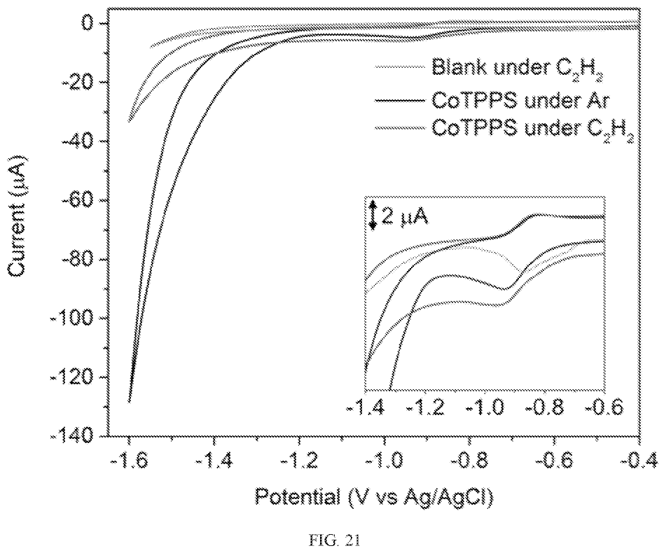

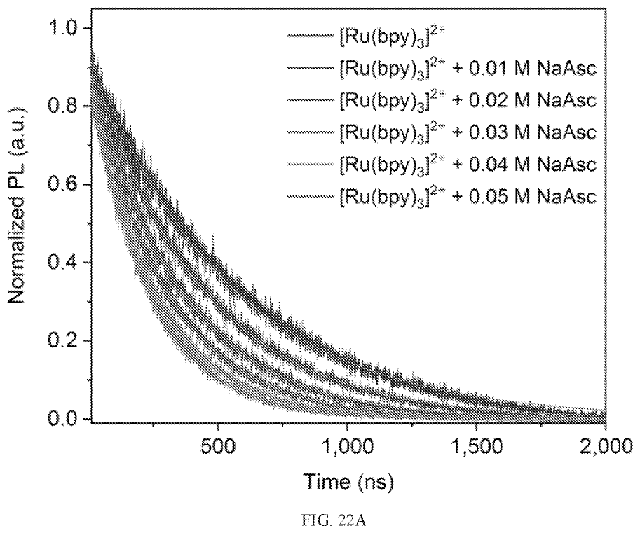

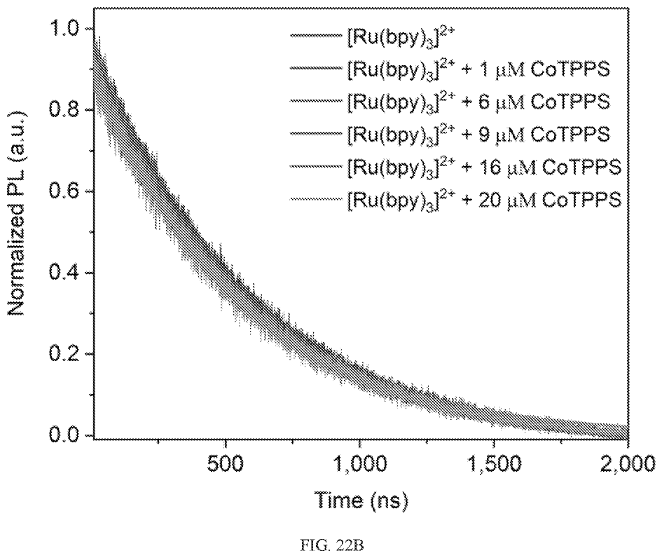

Based on these experimental results and the literature, a probable mechanism for the visible-light-driven reduction of C₂H₂ to C₂H₄ is diagrammed in FIG. 3A. The shift of the CoTPPS Soret band from 426 nm to 412 nm upon addition of NaAsc indicates that the starting [Co^{III}P(H₂O)(OH)] species is reduced spontaneously to [Co^{II}P(OH)] by NaAsc (FIG. 3B and FIG. 19). The importance of this reduction step is underscored by the observation that the starting [Co^{III}P(H₂O)(OH)] species is not reduced spontaneously to [Co^{II}P(OH)] by the other sacrificial donors and consequently no C₂H₄ production was observed when NaAsc was replaced with those donors (FIG. 12). Following light absorption by [Ru(bpy)₃]²⁺ (PS), the emission of the excited state [Ru(bpy)₃]²⁺* photosensitizer (PS*) is quenched by hole transfer to NaAsc to form [Ru(bpy)₂ (bpy′−)]⁺(PS⁻), followed by electron transfer from PS⁻Eₚ=−1.42 V vs Ag/AgCl) to [Co^{II}P(OH)](E_{1/2}=−0.87 V vs. Ag/AgCl) to form the low valent [Co^{I}P]⁻ species (FIGS. 20 and 21). The order of charge transfer reactions was determined by measuring the bimolecular rate constants for quenching of the photoluminescence of PS* by Stern-Volmer analysis (FIG. 3C and FIG. 22). These data show that photoinduced hole transfer to NaAsc is a factor of 1,000 faster than photoinduced electron transfer to CoTPPS. The nucleophilic attack by Co(l) species on one of the carbon atoms of the electrophilic C₂H₂, via a π complex, is followed by a rapid addition of a proton from water. In the final segments of the photocatalytic cycle (FIG. 3A), a second protonation of the Co—C bond by water yields C₂H₄ and [Co^{II}P(OH)] and re-starts the cycle.

It was confirmed that the Co(I) species is the active site to which C₂H₂ binds by adding NaBH₄, which is known to reduce the Co(III) of the CoTPPS to Co(I), to a CoTPPS solution without photosensitizer or light. Acetylene was converted to ethylene under these conditions, although H₂ is the major product (Table 1, entries 10 and 11).

A mechanism was ruled out in which the major intermediate under these experimental conditions is the cobalt hydride intermediate [Co^{III}—H], which would generate from the reaction of [Co^{I}P]⁻ with water and would then coordinate C₂H₂ and release C₂H₄ after a second protonation step. Based on a previous report of H₂ evolution using a CoTPPS catalyst at pH<8 in a phosphate buffer (Beyene et al. Sustain. Energy Fuels 2, 2036-2043 (2018)), formation of [Co^{III}—H] is believed to be disfavored due to the pH of the bicarbonate system (8.4, whereas the acid-base equilibrium constant between Co(I) and Co(III)—H is pKₐ=7.7) because the three-component system at pH≥8 did not evolve any detectable H₂ under C₂H₂(FIG. 7, Table 1, entry 1, and Table 5). H₂ was detected when the sample at pH≥8 was purged with Ar instead of C₂H₂ (FIG. 7, Table 1, entry 6 and Table 5). Although this result could indicate that C₂H₄ and H₂ products evolve from the same [Co^{III}—H] intermediate, C₂H₄ production and H₂ evolution do not share the same pH profile (FIG. 7, Table 1, entries 1.6 and Table 5), suggesting that the common [Co^{III}—H] intermediate is not the major pathway for the photoreduction to C₂H₄. It was observed that, while C₂H₄ production is relatively constant over the pH range 6.8-8.4 (FIG. 7 and Table 1, entry 1 and Table 5). H₂ evolution is more significant at more acidic pH and decreases at more basic pH when the sample was purged with Ar instead of C₂H₂(FIG. 7 and Table 1, entry 6 and Table 5). Also ruled out was the presence of radical intermediates because C₂H₄ formation was not affected by adding the radical trap TEMPO to the reaction mixture (Table 1, entry 12).

The geometry of the interaction between C₂H₂ and the CoTPPS catalyst was further specified by analyzing the stereoselectivity of the reduction reaction through gas-phase IR spectroscopy of the C₂H₂D₂ (m/z=30) product (FIG. 3D) obtained when the photoreduction was performed in D₂O solvent and using C₂H₂ (5 vol. %, He balance) as feedstock; The reaction of C₂H₂ in D₂O could lead to cis-, trans- or asymmetric-ethylene-d₂. The IR spectrum of the gaseous product had an intense absorption peak at 842 cm⁻¹ assigned to the non-planar vibration ω₇ of cis-C₂H₂D₂ (FIG. 3E). Another minor peak is attributable to ω₈C₂HD₃ (919 cm⁻¹) from the partially H/D exchanged acetylene substrate, and only barely visible are other two isomers of ethylene (asymmetric-C₂H₂D₂ and trans-C₂H₂D₂ at 943 and 987 cm⁻¹, respectively) (FIG. 3E). This result shows that the proton additions from water occur predominantly in a syn-manner (on one side of the triple bond of the substrate), which is consistent with the formation of a cis-alkene products from the cobalt(I)-catalyzed reduction of alkynes in alkaline media and consistent with the proposed mechanism.

It is suspected that the high selectivity of the three-component photocatalytic system originates in the better ability of the nucleophilic Co(I) species of the CoTPPS catalyst to coordinate highly electrophilic alkynes than less electrophilic alkenes (Table 5).

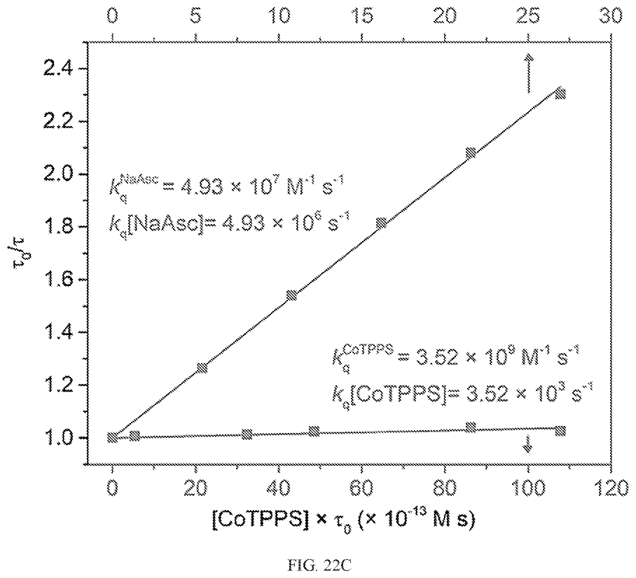

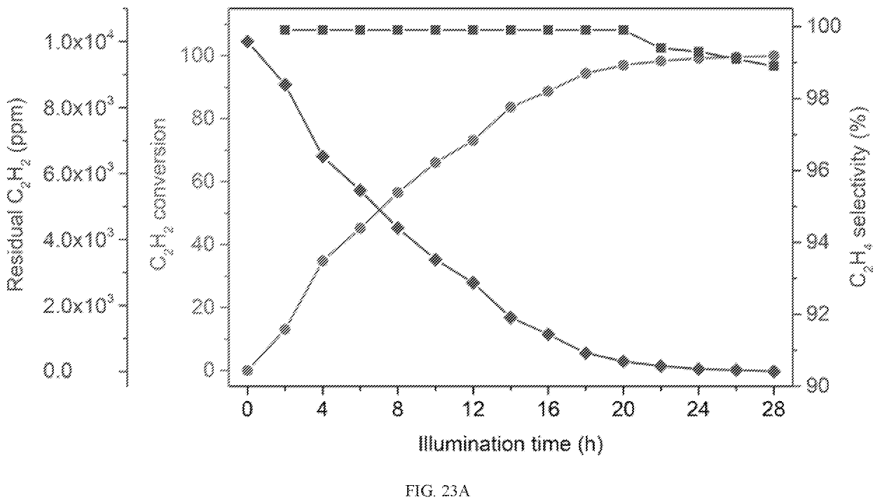

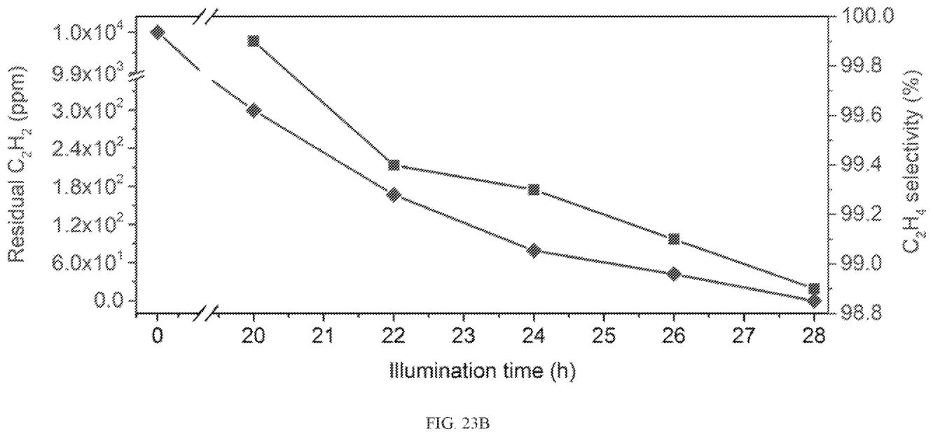

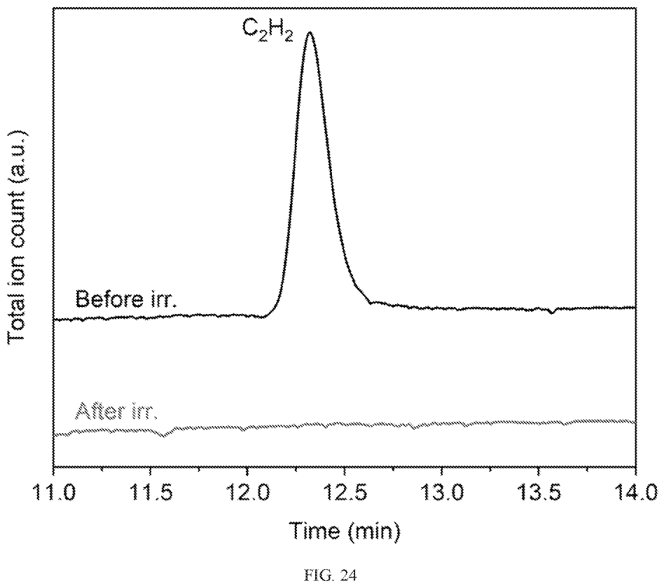

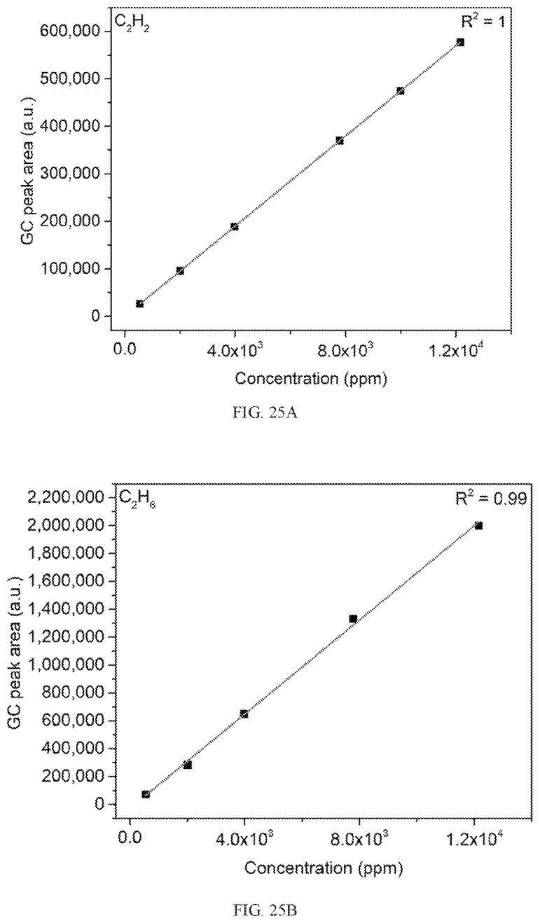

Importantly for potential industrial application of this process, the [Ru(bpy)₃]²⁺ sensitized CoTPPS system selectively photoreduces acetylene even in the presence of excess of ethylene (I vol. % C₂H₂, 30 vol. % C₂H₄. He balance). This ethylene/acetylene mixture is a typical industrial ethylene feed and requires a highly selective catalyst to eliminate ethane production. The system achieved near 100% conversion of C₂H₂ from this mixture with 98.9% selectivity for ethylene over ethane after 28 h of illumination (FIG. 4 and FIGS. 23-25). Improving the acetylene solubility in water is anticipated to decrease the illumination time necessary to achieve complete acetylene conversion.

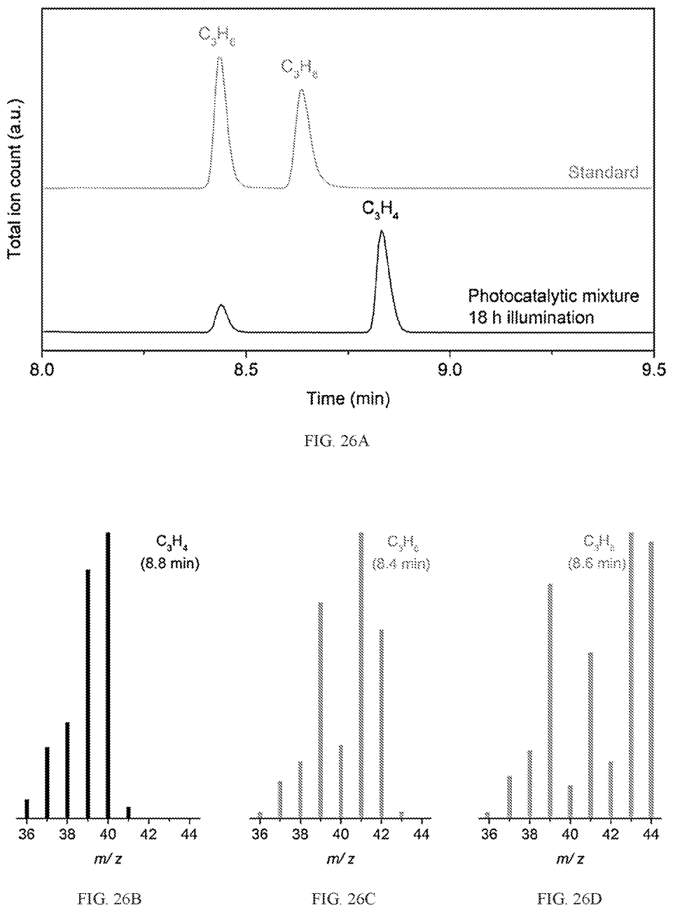

This photocatalytic system is not limited to conversion of acetylene. Illumination of the three-component system ([Ru(bpy)₃]²⁺, CoTPPS, NaAsc) and propyne (C₃H₄, 5 vol. %, He balance) produced propylene (C₃H₆) with >99% selectivity over propane (C₃H₈) (FIG. 26). This result demonstrates the suitability of this system for the isolation of a pure industrial propylene stream from propyne, a task not accomplished by steam cracking but necessary for the production of polymer-grade propylene, which together with ethylene accounts for ˜80% of global plastic demand.

Finally, CoTPPS can be replaced with a tetracarboxyphenyl cobalt porphyrin (CoTPPC) (Table 1, entry 13), which could be anchored to an electrode to exploit this catalytic cycle in a photoelectrochemical cell avoiding the use of a sacrificial electron donor.

1. A method of synthesizing an alkene compound from an alkyne compound, comprising:
providing a first mixture comprising a cobalt(III) porphyrin compound, a photosensitizer, a reducing agent, and water;
adding an alkyne compound to the first mixture to form a reaction mixture; and
illuminating the reaction mixture with light.

2. The method of claim 1, wherein the cobalt(III) porphyrin compound is selected from [{meso-tetra(4-sulfonatophenyl)porphyrinato}cobalt(III)] and [{meso-tetra(4-carboxyphenyl)porphyrinato}cobalt(III)].

3. The method of claim 2, wherein the cobalt(III) porphyrin compound is [{meso-tetra(4-sulfonatophenyl)porphyrinato}cobalt(III)].

4. The method of any one of claims 1-3, wherein the first mixture comprises the cobalt(III) porphyrin compound at a concentration of about 0.10 μM to about 50 μM.

5. The method of any one of claims 1-4, wherein the photosensitizer is selected from tris(2,2′-bipyridyl)dichlororuthenium(II) and mesoporous graphitic carbon nitride.

6. The method of claim 5, wherein the photosensitizer is tris(2,2′-bipyridyl)dichlororuthenium(II).

7. The method of claim 6, wherein the first mixture comprises the tris(2,2′-bipyridyl)dichlororuthenium(II) photosensitizer at a concentration of about 50 μM to about 500 μM.

8. The method of claim 5, wherein the photosensitizer is mesoporous graphitic carbon nitride.

9. The method of claim 8, wherein the first mixture comprises about 2.5 mg of the mesoporous graphitic carbon nitride.

10. The method of any one of claims 1-9, wherein the reducing agent is sodium ascorbate, or a mixture of sodium ascorbate and triethanolamine.

11. The method of any one of claims 1-10, wherein the first mixture comprises the reducing agent at a concentration of about 0.01 M to about 0.50 M.

12. The method of any one of claims 1-11, wherein the first mixture has a pH of about 8.0 to about 11.0.

13. The method of any one of claims 1-12, wherein the alkyne compound is a C₂-C₄ alkyne and the alkene compound is a C₂-C₄ alkene.

14. The method of claim 13, wherein the alkyne compound is acetylene and the alkene compound is ethylene.

15. The method of claim 13, wherein the alkyne compound is propyne and the alkene compound is propylene.

16. The method of any one of claims 1-15, wherein the light is selected from white light and blue light.

17. The method of any one of claims 1-16, comprising illuminating the reaction mixture with visible light from a light-emitting diode.

18. The method of any one of claims 1-17, wherein the first mixture is provided in a reaction vessel, and the step of adding the alkyne compound to the first mixture comprises adding the alkyne compound to the reaction vessel at a pressure of about 1 atm.

19. The method of any one of claims 1-18, wherein the method is conducted at ambient temperature.

20. A composition comprising a cobalt(III) porphyrin compound, a photosensitizer, a reducing agent, and water.

21. The composition of claim 20, wherein the cobalt(III) porphyrin compound is selected from [{meso-tetra(4-sulfonatophenyl)porphyrinato}cobalt(III)] and [{meso-tetra(4-carboxyphenyl)porphyrinato}cobalt(III)].

22. The composition of claim 21, wherein the cobalt(III) porphyrin compound is [{meso-tetra(4-sulfonatophenyl)porphyrinato}cobalt(III)].

23. The composition of any one of claims 20-22, wherein the composition comprises the cobalt(III) porphyrin compound at a concentration of about 0.10 μM to about 50 μM.

24. The composition of any one of claims 20-23, wherein the photosensitizer is selected from tris(2,2′-bipyridyl)dichlororuthenium(II) and mesoporous graphitic carbon nitride.

25. The composition of claim 24, wherein the photosensitizer is tris(2,2′-bipyridyl)dichlororuthenium(II).

26. The composition of claim 25, wherein the composition comprises the tris(2,2′-bipyridyl)dichlororuthenium(II) photosensitizer at a concentration of about 50 μM to about 500 μM.

27. The composition of claim 24, wherein the photosensitizer is mesoporous graphitic carbon nitride.

28. The composition of claim 27, wherein the composition comprises about 2.5 mg of the mesoporous graphitic carbon nitride.

29. The composition of any one of claims 20-28, wherein the reducing agent is sodium ascorbate, or a mixture of sodium ascorbate and triethanolamine.

30. The composition of any one of claims 20-29, wherein the composition comprises the reducing agent at a concentration of about 0.01 M to about 0.50 M.

31. The composition of any one of claims 20-30, wherein the composition has a pH of about 8.0 to about 11.0.

32. The composition of any one of claims 20-31, further comprising an alkyne compound.

33. The composition of claim 32, wherein the alkyne compound is a C₂-C₄ alkyne.

34. The composition of claim 33, wherein the alkyne compound is acetylene or propyne.

35. The composition of claim 34, wherein the alkyne compound is acetylene.
